(19) United States
(12) Patent Application Publication
(10) Pub. No.: US 2012/0213737 A1
(43) Pub. Date: Aug. 23, 2012

(54) COMPOSITIONS AND METHODS FOR THERAPEUTIC MEMBRANE REPAIR

(75) Inventors: Hua Zhu, Piscataway, NJ (US); Noah Weisleder, Elizabeth, NJ (US); Jianjie Ma, Belle Mead, NJ (US)

(73) Assignee: University of Medicine And Dentistry of New Jersey, Somerset, NJ (US)

(21) Appl. No.: 13/400,049

(22) Filed: Feb. 18, 2012

Related U.S. Application Data
(60) Provisional application No. 61/444,256, filed on Feb. 18, 2011.

(51) Int. Cl. A61K 31/7052; C12N 1/21; C12N 5/10; A61K 38/02; A61P 11/00; A61P 9/04; A61P 9/10; A61P 21/00; A61P 3/10; A61K 35/76; A61P 17/02

(52) U.S. Cl. 424/93.2; 514/44 R; 435/252.3; 435/325; 435/419; 514/1.1

(57) ABSTRACT

Disclosed herein are compositions comprising PTRF polypeptides, nucleic acids, and PTRF binding proteins useful for tissue regeneration and the treatment and prevention of disorders relating to cell membrane damage and repair.

### CROSS-REFERENCE TO RELATED APPLICATIONS

This application claims the benefit under 35 U.S.C. §119(e) of U.S. Provisional Application No. 61/444,256 filed Feb. 18, 2011, which is hereby incorporated by reference in its entirety for all purposes.

### STATEMENT REGARDING FEDERALLY SPONSORED RESEARCH

The U.S. Government has certain rights in this invention pursuant to the following grants: RO1-HL069000 awarded to Dr. Jianjie Ma by the United States National Institutes of Health (NIH).

### INCORPORATION BY REFERENCE

In compliance with 37 C.F.R. §1.52(e)(5), the sequence information filed electronically herewith, file name: Zhu_{—}2012utility_ST25.txt; size 38 KB; created on: Feb. 16, 2012; using PatentIn 3.5, and Checker 4.4.0 is hereby incorporated by reference in its entirety.

### FIELD OF THE INVENTION

This disclosure relates to polypeptides, nucleic acids encoding the same, antibodies that bind immunospecifically to polypeptides and associated methods of use.

### BACKGROUND

In response to external damage and internal degeneration, the cells of the body must repair the membrane surrounding each individual cell in order to maintain their function and the health of the organism. Defects in the ability of the cell to repair external membranes have been linked to many diseases, such as neurodegenerative diseases (Parkinson's Disease), heart attacks, heart failure and muscular dystrophy. Also, the muscle weakness and atrophy associated with various diseases, as well as the normal aging process, has been linked to altered membrane repair. In order for these cells to repair their membranes in response to acute damage, they make use of small packets of membrane that are inside of the cell, referred to as vesicles. These vesicles are normally found within the cell, but upon damage to the cell membrane, these vesicles move to the damage site and form a patch to maintain the cell integrity. Without this essential function, the cell can die and the cumulative effect of this cellular injury can eventually result in dysfunction of the tissue or organ.

Our previous studies show that MG53 is an essential component of the cell membrane repair machinery (Cai et al. 2009a), and defects in MG53-mediated membrane repair are linked to muscular dystrophy and cardiac dysfunction (Cai et al. 2009a; Cao et al.; Wang et al.).

Mutations in PTRF (polymerase I and transcript release factor (Jansa et al. 1998), also known as cavin-1, are associated with human disorders including lipodystrophy, muscular dystrophy and cardiac dysfunction (Dwianingsih et al.; Hayashi et al. 2009; Rajab et al.; Shastry et al.) but the precise mechanism is unknown (Bansal and Campbell 2004; Bansal et al. 2003; Doherty and McNally 2003). While several studies established PTRF regulates caveolae in the plasma membrane (Aboulaich et al. 2004; Hill et al. 2008; Liu et al. 2008), it is not known if PTRF participates in the membrane resealing process following acute injuries. Repair of cellular membranes in response to injury or other trauma is therapeutically relevant because such approaches can improve the regenerative capacity of various tissues.

### SUMMARY

The present description relates to the discovery that PTRF is involved in the process of membrane repair in a number of cell types, and by modulating its function the ability of these cells to repair their membranes can be modified and/or enhanced. Furthermore, the present description relates to the discovery that PTRF interacts with mitsugumin53 (MG53) in order to effectuate membrane resealing.

Thus, in certain aspects the description provides PTRF nucleic acids and polypeptides encoded from nucleic acids of the invention.

In additional aspects, the description provides compositions, for example, nucleic acids, which are useful for modulating (increasing or decreasing) the transcription or translation of target PTRF nucleic acids.

In another aspect, the description provides nucleic acids encoding cytoplasmic, nuclear, membrane bound, and secreted polypeptides; as well as vectors, host cells, antibodies, recombinant proteins, pseudopeptides, fusion proteins, chemical compounds, and methods for producing the same.

In certain aspects, the present invention also relates to compositions useful as therapeutics for treating and prevention of diseases and disorders. Therapeutic compositions of the invention comprise effective amounts of nucleic acids, including an interfering nucleic acids; nucleic acids encoding polypeptides corresponding to SEQ ID NOs. 1-4, including homologs, derivatives, and biologically active portions thereof (herein, “PTRF polypeptides”); PTRF polyepepties, including forms comprising psuedopeptides, peptide analogs and peptidomimetics; antibodies and antigen binding fragments and/or derivatives.

In certain additional aspects, the description provides compositions comprising any of the above in combination with another agent, e.g., an effective amount of MG53 interfering nucleic acids; nucleic acids encoding MG53 polypeptides; MG53 polypeptides and biologically active portions thereof; anti-MG53 antibodies and antigen binding fragments and/or derivatives.

In any of the aspects or embodiments described herein, the compositions may additionally include a pharmaceutically acceptable carrier, excipient or adjuvant.

As described herein, PTRF mediates the repair of damage to cellular membranes, and therefore, the targeting and modulating PTRF gene expression, polypeptide synthesis, activity or protein-protein interactions represent a novel therapeutic intervention for tissue repair.

In certain additional aspects the description provides compositions and methods related to the treatment of a pathological condition. In an exemplary embodiments, the methods comprise, for example, the administration of an effective amount of a therapeutic composition as described herein for the treatment of diabetes; promotion of wound healing; tissue repair and/or regeneration; for ameliorating surgical trauma; for treatment and/or prevention of age-related deficiencies in tissue repair that occur as a natural side-effect of the aging process; for treatment and/or prevention of injury to any type of muscle tissue, such as those occurring in subjects suffering from cardiovascular diseases and/or sports-related injuries; as well as the repair and regeneration of body tissues through cosmetic or personal care use.

In an additional aspect, the description provides methods for screening agents capable of modulating PTRF gene expression, activity and/or protein-protein interactions, wherein the agents identified are potential therapeutic candidates for treating any of the diseases or conditions recited herein.

The preceding general areas of utility are given by way of example only and are not intended to be limiting on the scope of the present disclosure and appended claims. Additional objects and advantages of the present invention will be appreciated by one of ordinary skill in the art in light of the instant claims, description, and examples. These additional objects and advantages are expressly included within the scope of the present invention.

### BRIEF DESCRIPTION OF THE DRAWINGS

FIG. 1. MG53 membrane repair function requires PTRF. A, microelectrode penetration of HeLa cells expressing GFP-MG53 results in translocation of MG53 at the injury site. B, MG53 disperses and leaks out of HepG2 cells upon the same treatment as A. arrowheads indicate the location of microelectrode penetration. C, Western blots show that PTRF is expressed in different mouse tissues, but not in liver and HepG2 cells. Transfection of RFP-PTRF in HepG2 cells produces protein expression (right panel). Tubulin serves as internal control (con).D, subcellular distribution of RFP, RFP-PTRF, and GFP-MG53 expressed in HepG2 cells, before and after treatment with 0.005% saponin. E, LDH release after glass microbead damage to HepG2 cells. Total LDH in the supernatant after mechanical damage minus the basal LDH level before damage was averaged from multiple experiments (mean±S.E., n=8). * indicates statistical difference with p<0.01 by analysis of variance.

FIG. 2. Modulating PTRF expression affects membrane repair in skeletal muscle. A, Western blot shows shRNA-mediated down-regulation of PTRF in FDB fibers. Con, control. B, FDB muscle fibers transfected with control plasmid show less FM1-43 dye entry following UV laser wounding (upper panels) as compared with those transfected with shRNA against PTRF (lower panels). Arrowheads indicate the location of laser wounding. Scale bar, 20 μm. C, summary data for panel B. Data represent mean±S.E., n=15. D, Western blot analysis shows overexpression of RFP-PTRF in dysferlin−/− and mg53−/− FDB muscle fibers. The endogenous levels of MG53, dysferlin, and PTRF in the different preparations are also shown. E, FDB muscle fibers from dysferlin−/− mice transfected with control plasmid (left) show excessive FM1-43 dye entry following UV laser wounding as compared with those transfected with RFP-PTRF (right; n=15).G, FDB muscle fibers from mg53−/−mice transfected with control plasmid (left) show similar FM1-43 dye entry following UV laser wounding as compared with those transfected with RFP-PTRF (right, n=15). Arrowheads indicate the location of laser wounding. F and H, summary data for panels E and G, respectively. Data represent mean±S.E., n=15.

FIG. 3. Mutant PTRF cannot rescue membrane repair defects in dysferlin−/−muscle. A, upper panels show mislocalization of RFP-531DelG in the nucleus of the dysferlin−/−muscle fiber (left, RFP fluorescence; right, overlay of bright field and fluorescence image). Lower panels show FM1-43 dye entry in the same FDB fiber following UV laser wounding. B, summary data from multiple experiments show that dysferlin−/−muscle fibers transfected with 531DelG-PTRF (red, n=12) display similar FM1-43 dye entry as those transfected with RFP as control (black, n=12), whereas muscle fibers transfected with the wild type PTRF display reduced FM1-43 dye entry (green, n=12). Data represent mean±S.E. C, RFP-531DelG expressed in HepG2 cells is localized to nucleus, and GFP-MG53 is present in both intracellular vesicles and plasma membrane (left panel). Fluorescent signals for RFP-531DelG and GFPMG53 disappear after treatment with 0.005% saponin (right panel, n=6). D, H1299 cells transfected with GFP-MG53 show trafficking of MG53 to the plasma membrane following treatment with 0.005% saponin. Scale bar, 20 μm. E, shows expression of wild type (wt) PTRF in skeletal muscle.

FIG. 4. PTRF anchors MG53 to membrane cholesterol for initiation of cell membrane repair. A, co-immunoprecipitation (IP) shows physical interaction between PTRF and MG53 in HeLa cells. B, lipid dot-blot analyses reveal PTRF can bind PS and cholesterol, but not PC. MBP-MG53 can bind PS, but not PC and cholesterol. As control, MBP-MBP does not show binding to PS, PC, or cholesterol. Co-incubation with PTRF leads to tethering of MG53 to cholesterol. WB, Western blot. C, treatment of FDB fibers with 5 mM MβCD(15 min at 37° C.) leads to fragility of the sarcolemmal membrane and defective resealing upon UV laser wounding. MβCD-treated FDB fibers always show contracture following UV irradiation (n=10). Scale bar, 20 μm. D, C2C12 cells injured with a microelectrode show minimum GFP-MG53 accumulation at the injury site, with 10 mM DTT and 0 mM Ca2+ present in the extracellular solution (upper panels). Preincubation with cholesterol (0.25 mM for 12 h) in the culture medium led to increased accumulation of GFP-MG53 at the injury site (lower panels). E, summary data for GFP-MG53 accumulation at injury sites from multiple experiments were presented (n=12). Data represent mean±S.E.

FIG. 5. The 531DelG mutation disrupts the interaction with MG53. Co-immunoprecipitation (IP) shows disruption of the physical interaction between the 531DelG PTRF mutant and MG53 in HeLa cells.

### DETAILED DESCRIPTION

Dynamic membrane repair is essential not only for long-term maintenance of cellular integrity but also for recovery from acute cell injury. Membrane repair defects have been linked to numerous disease states including muscular dystrophy, heart failure and neurodegeneration. Repair of the cell membrane requires intracellular vesicular trafficking that is associated with accumulation of vesicles at the plasma membrane. The present disclosure incorporates U.S. Pat. No. 7,981, by reference in its entirety for all purposes.

The present description relates to the discover that PTRF is an obligatory factor for MG53-mediated cell membrane repair, as cells lacking endogenous expression of PTRF are defective in resealing damaged membranes. As such, a new biological function for PTRF as an anchoring molecule for MG53 to initiate the cell membrane repair response is presently described. PTRF acts as a docking protein for MG53-mediated nucleation of the membrane repair machinery through binding exposed membrane cholesterol at the injury site. Overexpression of PTRF could not only rescue the membrane repair defects in liver cells, but also improve the membrane repair function in dysferlin−/−muscle fibers. The ineffective function of PTRF in the MG53 null background suggests that PTRF and MG53 must work together to allow membrane repair to occur. Cells lacking endogenous expression of PTRF show defective membrane resealing. RNAi-silencing of PTRF leads to defective membrane repair in muscle cells, and overexpression of PTRF can rescue membrane repair defects in dysferlin null muscle fibers. Mutation in PTRF associated with human disease alters PTRF localization in the nucleus and disrupts MG53-mediated membrane repair.

Our data reveal that membrane-delimited interaction between MG53 and PTRF at the cell injury site contributes to initiation of the cell membrane repair response. Without being limited by any particular theory, we propose that when cell membranes are disrupted, PTRF can recognize the exposed cholesterol at the injury site and tether MG53 and its associated intracellular vesicles to the damage site, facilitating formation of the membrane repair patch. Because many human diseases are associated with compromised membrane repair capacity, targeting the functional interaction between MG53 and PTRF or restoration of any disrupted MG53-PTRF interaction during disease presents an opportunity for treatment or prevention of tissue injury in human diseases.

Accordingly, in certain aspects the present disclosure provides biological polymers, including nucleic acids complementary to the gene (cDNA or RNA) encoding PTRF, nucleic acids capable of hybridizing to the same, nucleic acids encoding PTRF polypeptides and the PTRF polypeptides encoded thereby and biologically active portions thereof. For example, in certain aspects, the sequences are collectively referred to herein as “PTRF nucleic acids” or “PTRF polynucleotides,” and polypeptides are referred to as “PTRF polypeptides” or “PTRF proteins.” Unless indicated otherwise, “PTRF” is meant to refer to any of the sequences described herein.

The present invention relates to the discovery that vesicular fusion during acute membrane repair requires PTRF (see SEQ ID NOs.: 1-4). PTRF expression is necessary to allow intracellular vesicles trafficking to and fusion with the plasma membrane. Cells that are null for PTRF display defects in membrane repair in response to multiple stresses, including laser-induced or chemical-induced injury, muscle damage induced by exercise, and compromised recovery of muscle contractile function after fatigue. Thus, PTRF is a critical component of the vesicular trafficking events that underlie the acute repair and remodeling of cellular membranes.

In one aspect, the description provides an isolated and/or recombinant nucleic acid molecule encoding a PTRF polypeptide that includes a nucleic acid sequence that has at least 30%, 40%, 50%, 60%, 70%, 80%, 90% or 100% sequence identity to a nucleic acid disclosed in SEQ ID NO: 5, 6, 7, or 8. In certain embodiments, the isolated MG53 nucleic acid molecule will hybridize under stringent conditions to a nucleic acid sequence complementary to a nucleic acid molecule that includes a protein-coding sequence of a PTRF nucleic acid sequence. The invention also includes an isolated nucleic acid that encodes a PTRF polypeptide, or a biologically active fragment, homolog, analog, fusion protein, pseudopeptide, peptidomimetic or derivative thereof. For example, the nucleic acid can encode a polypeptide having at least 30%, 40%, 50%, 60%, 70%, 80%, 90% or 100% sequence identity to the amino acid sequence of SEQ ID NOS: 1, 2, 3, or 4. The nucleic acid can be, for example, an isolated genomic DNA fragment, a cDNA or mRNA molecule that includes the nucleic acid sequence of any of SEQ ID NOS: 5, 6, 7, or 8.

Also provided are oligonucleotides, e.g., an oligonucleotide which includes at least 6 contiguous nucleotides of a PTRF nucleic acid (e.g., SEQ ID NOs.: 5, 6, 7, and 8) or a complement of said oligonucleotide.

In another aspect, the invention includes pharmaceutical compositions that include therapeutically- or prophylactically-effective amounts of a therapeutic and a pharmaceutically-acceptable carrier. The therapeutic can be, e.g., a PTRF nucleic acid, for example, a peptide nucleic acid, a cDNA, or RNA, such as for example, a small inhibitory RNA or small hairpin RNA; a PTRF polypeptide; or an anti-PTRF immunoglobulin or derivative thereof. In a further aspect, the invention includes, in one or more containers, a therapeutically- or prophylactically-effective amount of this pharmaceutical composition.

In a further aspect, the invention includes a method of producing a polypeptide by culturing a cell that includes an endogenous or exogenously expressed PTRF nucleic acid, under conditions allowing for expression of the PTRF polypeptide encoded by the DNA. If desired, the PTRF polypeptide can then be recovered.

The description provides compositions and methods to identify specific cell or tissue types based on their expression of a PTRF nucleic acid, polypeptide or PTRF fusion polypeptide. For example, in certain embodiments the description provides fusion proteins comprising a “tag” or indicator portion and a PTRF portion. In certain aspects the tag or indicator portion can be a peptide adapted for purification purposes, for example, a secretion signal peptide, a FLAG tag, 6×His tag, or the like. In other embodiments, the tag peptide comprises a peptide adapted for providing a signal such as an antibody epitope or a fluorescent peptide. Still other embodiments include the fusion of the PTRF with a peptide that is adapted for mediating subcellular localization or enhance translocation across a cellular membrane, for example, a TAT fusion protein from the HIV virus or a secretion signal peptide. In an additional embodiment, the description provides a fusion protein (and nucleic acid encoding the same) comprising PTRF and mitsugumin53 (MG53),

Also included in the invention is a method of detecting the presence of a PTRF nucleic acid molecule in a sample by contacting the sample with a PTRF nucleic acid probe or primer, and detecting whether the nucleic acid probe or primer bound to a PTRF nucleic acid molecule in the sample.

Also within the scope of the invention is the use of a therapeutic of the invention in the manufacture of a medicament for treating or preventing disorders or syndromes including, e.g., cardiovascular disease, cardiomyopathy, atherosclerosis, ulcers, wounds, lesions, cuts, abrasions, oxidative damage, age-related tissue degeneration, surgically related lesions, burns, muscle weakness, muscle atrophy, connective tissue disorders, idiopathic thrombocytopenic purpura, heart failure, secondary pathologies caused by heart failure and hypertension, hypotension, angina pectoris, myocardial infarction, tuberous sclerosis, scleroderma, inflammation, viral pathogenesis, aging-related disorders, multiple sclerosis, inflammatory bowel diseases, wound repair, heart attacks, heart failure, muscular dystrophy, bed sores, diabetic ulcers, oxidative damage, and tissue damage such as sinusitis or mucositis, wrinkles, eczema or dermatitis, dry skin, obesity, diabetes, and/or other pathologies and disorders of the like.

In certain aspects, the description provides a therapeutic composition comprising a pharmaceutically acceptable carrier and/or excipient and an effective amount of PTRF nucleic acid selected from the group consisting of: (i) a nucleic acid having at least 30%, 40%, 50%, 60%, 70%, 80%, 90% or 100% sequence identity to a nucleic acid disclosed in SEQ ID NOs.: 5, 6, 7, or 8; (ii) a nucleic acid that is complementary to at least a portion of a nucleic acid of (i); (iii) a nucleic acid that is capable of hybridizing to a nucleic acid of (i); (iv) a nucleic acid capable of hybridizing to a nucleic acid of (ii) or (iii); (v) a nucleic acid encoding a polypeptide having at least 30%, 40%, 50%, 60%, 70%, 80%, 90% or 100% sequence identity to an amino acid of and (vi) combinations thereof.

In still other embodiments, the invention comprises therapeutic compositions useful as a surgical adjuvant. In any of the embodiments described herein, the surgical adjuvant composition of the invention can be used or applied as a stand alone therapeutic directly to the surgical site or it can be integrally associated with a surgical or medical implement, for example, the therapeutic of the invention may be conjugated to a polymer-based stent, tube or other implantable device, such that the therapeutic diffuses to the site of action in a controlled manner to accelerate healing and/or to minimize trauma from an invasive surgical procedure. In another embodiment, the therapeutic composition of the invention is applied as, for example, a film or coating to the medical implement such that the therapeutic diffuses into the blood stream or surrounding tissues and/or wears away, and is thereby delivered directly to the site of tissue damage; minimizing or ameliorating the amount of cellular damage that occurs due to the use of the surgical implement.

In still other embodiments, the invention comprises methods for the treatment and/or prevention of deficiencies in tissue repair that occur as a natural side-effect of the aging process (e.g., skin rejuvenation, receding gums, bone degeneration, arthritis, Alzheimer's, Parkinson's, and the like). In certain aspects of this embodiment, the invention comprises administering an effective amount of a therapeutic composition of the invention to a subject suffering from age-related deficiencies in tissue repair capacity, tissue integrity, and/or tissue elasticity. In certain embodiments, the age-related deficiency is at least one of wrinkles, crows feet, facial lines, pot marks, scars, fibroids, sun spots, and the like, or combinations thereof.

Furthermore, the invention encompasses methods for the treatment and/or prevention of any type of muscle or vascular cell/tissue injury, for example, tissue injury that occurs as a result of cardiovascular disease, for example, myocardial infraction; or rigorous physical activity, for example, sports-related injuries, comprising administering an effective amount of the therapeutic of the invention to a subject in need thereof.

In still other embodiments, the invention comprises a cosmetic composition useful for the repair, regeneration, or restoration of body tissues comprising the therapeutic of the invention and a cosmetically suitable carrier or excipient. In one aspect of this embodiment, the invention encompasses a method of enhancing the appearance of skin comprising administering an effective amount of the therapeutic composition of the invention in a cosmetic to a subject.

In any of the aspects or embodiments described herein, the therapeutic composition can be in any pharmaceutically acceptable form and administered by any pharmaceutically acceptable route, for example, the therapeutic composition can be administered as an oral dosage, either single daily dose or unitary dosage form, for the treatment of a muscle damage due to a myocardial infarction, sclerotic lesion, or muscle tear due to sports-related activity to promote the regeneration and repair of the damaged muscle tissue. Such pharmaceutically acceptable carriers and excipients and methods of administration will be readily apparent to those of skill in the art.

The description provides nucleic acids, including interfering nucleic acids, and polypeptides encoding PTRF interacting proteins, for example, MG53 (SEQ ID NO: 9) polypeptides and homologs thereof; psuedopeptides and peptidomimetics; as well as compounds that can modulate the activity of MG53 or its intermolecular interactions with PTRF. Therefore, in additional aspects, the present invention encompasses methods for the targeting of PTRF and/or MG53 gene expression, activity, and/or intermolecular interactions for the treatment and/or prevention of a disease or disorder in a subject, for example, for the promotion of tissue repair as described above.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. In addition, a cDNA encoding PTRF may be useful in gene therapy, and PTRF may be useful when administered to a subject in need thereof. By way of non-limiting example, the compositions of the present invention will have efficacy for treatment of patients suffering from the diseases and disorders disclosed above and/or other pathologies and disorders of the like.

The description further provides a method for screening for a modulator of disorders or syndromes including, e.g., the diseases and disorders disclosed above and/or other pathologies and disorders of the like. The method includes contacting a test compound with a PTRF polypeptide and determining if the test compound binds to said PTRF polypeptide. Binding of the test compound to the PTRF polypeptide indicates the test compound is a modulator of activity, or of latency or predisposition to the aforementioned disorders or syndromes.

The description also provides a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a PTRF nucleic acid. Expression or activity of PTRF polypeptide is then measured in the test animal, as is expression or activity of the protein in a control animal which recombinantly-expresses PTRF polypeptide and is not at increased risk for the disorder or syndrome. Next, the expression of PTRF polypeptide in both the test animal and the control animal is compared. A change in the activity of PTRF polypeptide in the test animal relative to the control animal indicates the test compound is a modulator of latency of the disorder or syndrome.

In yet another aspect, the description provides a method for determining the presence of or predisposition to a disease associated with altered levels of a PTRF polypeptide, a PTRF nucleic acid, or both, in a subject (e.g., a human subject). The method includes measuring the amount of the PTRF polypeptide in a test sample from the subject and comparing the amount of the polypeptide in the test sample to the amount of the PTRF polypeptide present in a control sample. An alteration in the level of the PTRF polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various disorders as well as to determine the stage of particular disorders.

In a further aspect, the description provides a method of treating or preventing a pathological condition associated with a disorder in a mammal by administering to the subject a PTRF polypeptide, a PTRF nucleic acid, or a PTRF-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the description provides a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

As used herein, the term “PTRF antagonist” or “antagonist of PTRF” is used generally to refer to an agent capable of direct or indirect inhibition of MG53 expression, translation, and/or activity. Also, as used herein “PTRF receptor” relates generally to any protein or fragment thereof capable of undergoing binding to a PTRF protein.

In certain aspects, the modulation of PTRF activity is accomplished by, for example, the use of or modulation of PTRF binding partners, i.e., factors that bind to PTRF and neutralize its biological activities, such as neutralizing anti-PTRF, PTRF receptors (for example, MG53), PTRF receptor fragments, and PTRF receptor analogs; the use of PTRF-receptor antagonists, such as anti-MG533 antibodies, pseudopeptides, peptide analogs or peptidomimetics that bind and disrupt the PTRF-receptor interaction; small molecules that inhibit PTRF activity or intermolecular interactions, or alter the normal configuration of PTRF, or inhibit productive PTRF/PTRF-receptor binding; or the use of nucleotide sequences derived from PTRF gene and/or PTRF receptor gene, including coding, non-coding, and/or regulatory sequences to prevent or reduce PTRF expression by, for example, antisense, ribozyme, and/or triple helix approaches.

In another aspect, the present invention features a nucleic acid molecule, such as a decoy RNA, dsRNA, siRNA, shRNA, micro RNA, aptamers, antisense nucleic acid molecules, which down regulates expression of a sequence encoding PTRF or a PTRF-receptor, for example, MG53. In an embodiment, a nucleic acid molecule of the invention is adapted to treat and/or prevent tissue damage and promote tissue repair. In another embodiment, a nucleic acid molecule of the invention has an endonuclease activity or is a component of a nuclease complex, and cleaves RNA having a PTRF or a PTRF receptor nucleic acid sequence.

In one embodiment, a nucleic acid molecule of the invention comprises between 12 and 100 bases complementary to RNA having a PTRF or a PTRF-receptor nucleic acid sequence. In another embodiment, a nucleic acid molecule of the invention comprises between 14 and 24 bases complementary to RNA having a PTRF or a PTRF-receptor nucleic acid sequence. In any embodiment described herein, the nucleic acid molecule can be synthesized chemically according to methods well known in the art.

In another aspect the present invention provides a kit comprising a suitable container, the active agent capable of inhibiting PTRF activity, expression or binding in a pharmaceutically acceptable form disposed therein, and instructions for its use.

In another aspect, the invention relates to a method for diagnosing or monitoring disorder or disease or progression comprising detecting for the presence of a nucleotide polymorphism in the PTRF or a PTRF-receptor structural gene associated with the disease, through the detection of the expression level of a PTRF or a PTRF-receptor gene or protein or both. Polymorphisms have been identified that correlate with disease severity. (See, Zhong et al., Simultaneous detection of microsatellite repeats and SNPs in the macrophage migration inhibitory factor (MG53) gene by thin-film biosensor chips and application to rural field studies. Nucleic Acids Res. 2005 Aug. 2; 33(13):e121; Donn et al., A functional promoter haplotype of macrophage migration inhibitory factor is linked and associated with juvenile idiopathic arthritis. Arthritis Rheum. 2004 May; 50(5):1604-10; all of which are incorporated herein by reference in their entirety for all purposes.). As used herein, “PTRF or a PTRF-receptor gene” or “PTRF or a PTRF-receptor structural gene” may include the 5′ UTR, 3′ UTR, promoter sequences, enhancer sequences, intronic and exonic DNA of the PTRF or a PTRF-receptor gene as well as the PTRF or a PTRF-receptor gene mRNA or cDNA sequence.

As one of ordinary skill will comprehend, the PTRF or a PTRF-receptor gene polymorphisms associated with tissue repair disorders, and hence useful as diagnostic markers according to the methods of the invention may appear in any of the previously named nucleic acid regions. Techniques for the identification and monitoring of polymorphisms are known in the art and are discussed in detail in U.S. Pat. No. 6,905,827 to Wohlgemuth, which is incorporated herein by reference in its entirety for all purposes.

In certain aspects, the description provides methods of detecting gene expression or polymorphisms with one or more DNA molecules wherein the one or more DNA molecules has a nucleotide sequence which detects expression of a gene corresponding to the oligonucleotides depicted in the Sequence Listing. In one format, the oligonucleotide detects expression of a gene that is differentially expressed. The gene expression system may be a candidate library, a diagnostic agent, a diagnostic oligonucleotide set or a diagnostic probe set. The DNA molecules may be genomic DNA, RNA, protein nucleic acid (PNA), cDNA or synthetic oligonucleotides. Following the procedures taught herein, one can identify sequences of interest for analyzing gene expression or polymorphisms. Such sequences may be predictive of a disease state.

Diagnostic Oligonucleotides of the Invention

As used herein, the term “oligonucleotide molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

In certain aspects, the invention relates to diagnostic oligonucleotides and diagnostic oligonucleotide set(s), for which a correlation exists between the health status of an individual, and the individual's expression of RNA or protein products corresponding to the nucleotide sequence. In some instances, only one oligonucleotide is necessary for such detection. Members of a diagnostic oligonucleotide set may be identified by any means capable of detecting expression or a polymorphism of RNA or protein products, including but not limited to differential expression screening, PCR, RT-PCR, SAGE analysis, high-throughput sequencing, microarrays, liquid or other arrays, protein-based methods (e.g., western blotting, proteomics, mass-spectrometry, and other methods described herein), and data mining methods, as further described herein.

In the context of the invention, nucleic acids and/or proteins are manipulated according to well known molecular biology techniques. Detailed protocols for numerous such procedures are described in, e.g., in Ausubel et al. Current Protocols in Molecular Biology (supplemented through 2000) John Wiley & Sons, New York (“Ausubel”); Sambrook et al. Molecular Cloning-A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”).

Genotyping

In addition to, or in conjunction with the correlation of expression profiles and clinical data, it is often desirable to correlate expression patterns with the subject's genotype at one or more genetic loci or to correlate both expression profiles and genetic loci data with clinical data. The selected loci can be, for example, chromosomal loci corresponding to one or more member of the candidate library, polymorphic alleles for marker loci, or alternative disease related loci (not contributing to the candidate library) known to be, or putatively associated with, a disease (or disease criterion). Indeed, it will be appreciated, that where a (polymorphic) allele at a locus is linked to a disease (or to a predisposition to a disease), the presence of the allele can itself be a disease criterion.

Numerous well known methods exist for evaluating the genotype of an individual, including southern analysis, restriction fragment length polymorphism (RFLP) analysis, polymerase chain reaction (PCR), amplification length polymorphism (AFLP) analysis, single stranded conformation polymorphism (SSCP) analysis, single nucleotide polymorphism (SNP) analysis (e.g., via PCR, Taqman or molecular beacons), among many other useful methods. Many such procedures are readily adaptable to high throughput and/or automated (or semi-automated) sample preparation and analysis methods. Most, can be performed on nucleic acid samples recovered via simple procedures from the same sample as yielded the material for expression profiling. Exemplary techniques are described in, e.g., Sambrook, and Ausubel, supra.

The invention also features nucleic acid molecules, for example enzymatic nucleic acid molecules, antisense nucleic acid molecules, decoys, double stranded RNA, triplex oligonucleotides, and/or aptamers, and methods to modulate gene expression of, for example, genes encoding a PTRF protein, a PTRF-receptor or a PTRF-receptor binding protein. In particular, the instant invention features nucleic-acid based molecules and methods to modulate the expression of a PTRF or a PTRF-receptor protein.

The invention features one or more enzymatic nucleic acid-based molecules and methods that independently or in combination modulate the expression of gene(s) encoding a PTRF or a PTRF-receptor, for example, MG53.

The description below of the various aspects and embodiments is provided with reference to the exemplary PTRF or a PTRF-receptor genes. However, the various aspects and embodiments are also directed to genes which encode homologs, orthologs, and paralogs of other PTRF or a PTRF-receptor proteins, and PTRF or a PTRF-receptor genes and include all isoforms, splice variants, and polymorphisms. Those additional genes can be analyzed for target sites using the methods described for PTRF or a PTRF-receptor genes. Thus, the inhibition and the effects of such inhibition of the other genes can be performed as described herein.

By “down-regulate” it is meant that the expression of the gene, or level of RNAs or equivalent RNAs encoding one or more proteins, or activity of one or more proteins, such as PTRF or a PTRF-receptor genes, is reduced below that observed in the absence of the nucleic acid molecules of the invention. In one embodiment, inhibition or down-regulation with enzymatic nucleic acid molecule preferably is below that level observed in the presence of an enzymatically inactive or attenuated molecule that is able to bind to the same site on the target RNA, but is unable to cleave that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of PTRF or a PTRF-receptor genes with the nucleic acid molecule of the instant invention is greater in the presence of the nucleic acid molecule than in its absence.

By “up-regulate” is meant that the expression of the gene, or level of RNAs or equivalent RNAs encoding one or more protein subunits, or activity of one or more protein subunits, such as PTRF or a PTRF-receptor genes, is greater than that observed in the absence of the nucleic acid molecules of the invention. For example, the expression of a gene, such as PTRF or a PTRF-receptor genes, can be increased in order to treat, prevent, ameliorate, or modulate a pathological condition caused or exacerbated by an absence or low level of gene expression. In one embodiment the invention relates to a method for treating or preventing bladder over activity by up-regulating the expression, release, and/or activity of a PTRF or a PTRF-receptor genes.

By “modulate” is meant that the expression of the gene, or level of RNAs or equivalent RNAs encoding one or more proteins, or activity of one or more proteins is up-regulated or down-regulated, such that the expression, level, or activity is greater than or less than that observed in the absence of the nucleic acid molecules of the invention.

By “gene” it is meant a nucleic acid that encodes RNA, for example, nucleic acid sequences including but not limited to a segment encoding a polypeptide.

“Complementarity” refers to the ability of a nucleic acid to form hydrogen bond(s) with another RNA sequence by either traditional Watson-Crick or other non-traditional types.

By “RNA” is meant a molecule comprising at least one ribonucleotide residue. By “ribonucleotide” or “2′-OH” is meant a nucleotide with a hydroxyl group at the 2′ position of a D-ribo-furanose moiety.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N-6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra).

By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “enzymatic nucleic acid molecule” it is meant a nucleic acid molecule which has complementarity in a substrate binding region to a specified gene target, and also has or mediates an enzymatic activity which is active to specifically cleave target RNA. That is, the enzymatic nucleic acid molecule is able to intermolecularly cleave RNA, alone or as a component of an enzymatic complex, and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092 2096; Hammann et al., 1999, Antisense and Nucleic Acid Drug Dev., 9, 25 31). The nucleic acids can be modified at the base, sugar, and/or phosphate groups. The term “enzymatic nucleic acid” is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, siRNA, micro RNA, short hairpin RNA, endoribonuclease, RNA-induced silencing complexes, endonuclease, minizyme, leadzyme, oligozyme or DNA enzyme. All of these terminologies describe nucleic acid molecules with enzymatic activity.

The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

Several varieties of enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

By “nucleic acid molecule” as used herein is meant a molecule having nucleotides. The nucleic acid can be single, double, or multiple stranded and can comprise modified or unmodified nucleotides or non-nucleotides or various mixtures and combinations thereof.

By “equivalent” or “related” RNA to PTRF or a PTRF-receptor gene is meant to include those naturally occurring RNA molecules having homology (partial or complete) to PTRF or PTRF-receptor genes encoding for proteins with similar function as PTRF or a PTRF-receptor proteins in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like. By “homology” is meant the nucleotide sequence of two or more nucleic acid molecules is partially or completely identical. In certain embodiments the homolgous nucleic acid has 30%, 40%, 50%, 60%, 70%, 80%, 90%, or 95% homology to a PTRF or a PTRF-receptor gene.

By “antisense nucleic acid”, it is meant a non-enzymatic nucleic acid molecule that binds to target RNA by means of RNA—RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop or hairpin, and/or an antisense molecule can bind such that the antisense molecule forms a loop or hairpin. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol, 40, 1-49, which are incorporated herein by reference in their entirety. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

Long double-stranded RNAs (dsRNAs; typically >200 nt) can be used to silence the expression of target genes in a variety of organisms and cell types (e.g., worms, fruit flies, and plants). Upon introduction, the long dsRNAs enter a cellular pathway that is commonly referred to as the RNA interference (RNAi) pathway. First, the dsRNAs get processed into 20-25 nucleotide (nt) small interfering RNAs (siRNAs) by an RNase III-like enzyme called Dicer (initiation step). Then, the siRNAs assemble into endoribonuclease-containing complexes known as RNA-induced silencing complexes (RISCs), unwinding in the process. The siRNA strands subsequently guide the RISCs to complementary RNA molecules, where they cleave and destroy the cognate RNA (effecter step). Cleavage of cognate RNA takes place near the middle of the region bound by the siRNA strand. In mammalian cells, introduction of long dsRNA (>30 nt) initiates a potent antiviral response, exemplified by nonspecific inhibition of protein synthesis and RNA degradation. The mammalian antiviral response can be bypassed, however, by the introduction or expression of siRNAs.

Injection and transfection of dsRNA into cells and organisms has been the main method of delivery of siRNA. And while the silencing effect lasts for several days and does appear to be transferred to daughter cells, it does eventually diminish. Recently, however, a number of groups have developed expression vectors to continually express siRNAs in transiently and stably transfected mammalian cells. (See, e.g., Brummelkamp T R, Bernards R, and Agami R. (2002). A system for stable expression of short interfering RNAs in mammalian cells. Science 296:550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052, which are herein incorporated by reference in their entirety).

Some vectors have been engineered to express small hairpin RNAs (shRNAs), which get processed in vivo into siRNAs-like molecules capable of carrying out gene-specific silencing. The vectors contain the shRNA sequence between a polymerase III (pol III) promoter (e.g., U6 or H1 promoters) and a 4-5 thymidine transcription termination site. The transcript is terminated at position 2 of the termination site (pol III transcripts naturally lack poly(A) tails) and then folds into a stem-loop structure with 3′ UU-overhangs. The ends of the shRNAs are processed in vivo, converting the shRNAs into ˜21 nt siRNA-like molecules, which in turn initiate RNAi. This latter finding correlates with recent experiments in C. elegans, Drosophila, plants and Trypanosomes, where RNAi has been induced by an RNA molecule that folds into a stem-loop structure. The use of siRNA vectors and expression systems is known and are commercially available from Ambion, Inc.® (Austin Tex.), Lentigen, Inc. (Baltimore, Md.), Panomics (Fremont, Calif.), and Sigma-Aldrich (ST. Louis, Mo.).

In another aspect of the invention, enzymatic nucleic acid molecules or antisense molecules that interact with target RNA molecules, and down-regulate PTRF or a PTRF-receptor gene activity are expressed from transcription units inserted into DNA or RNA vectors. The recombinant vectors are preferably DNA plasmids or viral vectors. Enzymatic nucleic acid molecule or antisense expressing viral vectors can be constructed based on, but not limited to, lenti virus, cytomegalovirus, adeno-associated virus, retrovirus, adenovirus, or alphavirus. Preferably, the recombinant vectors capable of expressing the enzymatic nucleic acid molecules or antisense are delivered, and persist in target cells. Alternatively, viral vectors can be used that provide for expression of enzymatic nucleic acid molecules or antisense. Such vectors can be repeatedly administered as necessary. Once expressed, the enzymatic nucleic acid molecules or antisense bind to the target RNA and down-regulate its function or expression. Delivery of enzymatic nucleic acid molecule or antisense expressing vectors can be systemic, such as by intravenous or intramuscular administration, by administration to target cells explanted from the patient or subject followed by reintroduction into the patient or subject, or by any other means that would allow for introduction into the desired target cell. Antisense DNA can be expressed via the use of a single stranded DNA intracellular expression vector.

By “vectors” is meant any nucleic acid-based technique used to deliver a desired nucleic acid, for example, bacterial plasmid, viral nucleic acid, HAC, BAC, and the like.

The nucleic acid molecules of the instant invention, individually, or in combination or in conjunction with other drugs, can be used to treat diseases or conditions discussed above. For example, the subject can be treated, or other appropriate cells can be treated, as is evident to those skilled in the art, individually or in combination with one or more drugs under conditions suitable for the treatment.

The use of specially designed vector constructs for inducing RNA interference has numerous advantages over oligonucleotide-based techniques. The most significant advantages are stability, and induced transcription via inducible promoters. Promoter regions in the vector ensure that shRNA transcripts are constantly expressed, maintaining cellular levels at all times. Thus, the duration of the effect is not as transient as with injected RNA, which usually lasts no longer than a few days. And by using expression constructs instead of oligo injection, it is possible to perform multi-generational studies of gene knockdown because the vector can become a permanent fixture in the cell line.

By “triplex forming oligonucleotides” or “triplex oligonucleotide” is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Cum Med. Chem., 7, 17-37; Praseuth et. al., 2000, Biochim. Biophys. Acta, 1489, 181-206).

By “double stranded RNA” or “dsRNA” is meant a double stranded RNA that matches a predetermined gene sequence that is capable of activating cellular enzymes that degrade the corresponding messenger RNA transcripts of the gene. These dsRNAs are referred to as short intervening RNA (siRNA) and can be used to inhibit gene expression. see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914.

In one embodiment of the present invention, a nucleic acid molecule of the instant invention can be between about 10 and 100 nucleotides in length. For example, enzymatic nucleic acid molecules of the invention are preferably between about 15 and 50 nucleotides in length, more preferably between about 25 and 40 nucleotides in length (for example see Jarvis et al., 1996, J. Biol. Chem., 271, 29107 29112). Exemplary antisense molecules of the invention are preferably between about 15 and 75 nucleotides in length, more preferably between about 20 and 35 nucleotides in length (see for example Woolf et al., 1992, PNAS., 89, 7305 7309; Milner et al., 1997, Nature Biotechnology, 15, 537 541). Exemplary triplex forming oligonucleotide molecules of the invention are preferably between about 10 and 40 nucleotides in length, more preferably between about 12 and 25 nucleotides in length (see for example Maher et al, 1990, Biochemistry, 29, 8820 8826; Strobel and Dervan, 1990, Science, 249, 73 75). Those skilled in the art will recognize that all that is required is that the nucleic acid molecule be of sufficient length and suitable conformation for the nucleic acid molecule to interact with its target and/or catalyze a reaction contemplated herein. The length of the nucleic acid molecules of the instant invention are not limiting within the general limits stated. Preferably, a nucleic acid molecule that modulates, for example, down-regulates PTRF or a PTRF-receptor gene expression comprises between 12 and 100 bases complementary to a RNA molecule of a PTRF or a PTRF-receptor gene.

The invention provides a method for producing a class of nucleic acid-based gene modulating agents which exhibit a high degree of specificity for the RNA of a desired target. For example, the enzymatic nucleic acid molecule is preferably targeted to a highly conserved sequence region of target RNAs encoding a PTRF or a PTRF-receptor gene such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

“Derivatives” are compositions formed from the native compounds either directly, by modification, or by partial substitution.

“Analogs” are nucleic acid sequences or amino acid sequences that have a structure similar to, but not identical to, the native compound.

Derivatives or analogs of the nucleic acids or proteins of the invention include, but are not limited to, molecules comprising regions that are substantially homologous to the nucleic acids or proteins of the invention, in various embodiments, by at least about 30%, 40%, 50%, 60%, 70%, 80%, 90%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins of the invention under stringent, moderately stringent, or low stringent conditions. See e.g. Ausubel, et al., CURRENT PROTOCOLS IN MOLECULAR BIOLOGY, John Wiley & Sons, New York, N.Y., 1993. Nucleic acid derivatives and modifications include those obtained by gene replacement, site-specific mutation, deletion, insertion, recombination, repair, shuffling, endonuclease digestion, PCR, subcloning, and related techniques.

“Homologs” can be naturally occurring, or created by artificial synthesis of one or more nucleic acids having related sequences, or by modification of one or more nucleic acid to produce related nucleic acids. Nucleic acids are homologous when they are derived, naturally or artificially, from a common ancestor sequence (e.g., orthologs or paralogs). If the homology between two nucleic acids is not expressly described, homology can be inferred by a nucleic acid comparison between two or more sequences. If the sequences demonstrate some degree of sequence similarity, for example, greater than about 30% at the primary amino acid structure level, it is concluded that they share a common ancestor. For purposes of the present invention, genes are homologous if the nucleic acid sequences are sufficiently similar to allow recombination and/or hybridization under low stringency conditions.

As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA.

Furthermore, one of ordinary skill will recognize that “conservative mutations” also include the substitution, deletion or addition of nucleic acids that alter, add or delete a single amino acid or a small number of amino acids in a coding sequence where the nucleic acid alterations result in the substitution of a chemically similar amino acid. Amino acids that may serve as conservative substitutions for each other include the following: Basic: Arginine (R), Lysine (K), Histidine (H); Acidic: Aspartic acid (D), Glutamic acid (E), Asparagine (N), Glutamine (Q); hydrophilic: Glycine (G), Alanine (A), Valine (V), Leucine (L), Isoleucine (I); Hydrophobic: Phenylalanine (F), Tyrosine (Y), Tryptophan (W); Sulfur-containing: Methionine (M), Cysteine (C). In addition, sequences that differ by conservative variations are generally homologous.

As used herein, “fragments” are defined as sequences of at least 6 (contiguous) nucleic acids or at least 4 (contiguous) amino acids, a length sufficient to allow for specific hybridization in the case of nucleic acids or for specific recognition of an epitope in the case of amino acids, and are at most some portion less than a full length sequence.

The term “host cell” includes a cell that might be used to carry a heterologous nucleic acid, or expresses a peptide or protein encoded by a heterologous nucleic acid. A host cell can contain genes that are not found within the native (non-recombinant) form of the cell, genes found in the native form of the cell where the genes are modified and re-introduced into the cell by artificial means, or a nucleic acid endogenous to the cell that has been artificially modified without removing the nucleic acid from the cell. A host cell may be eukaryotic or prokaryotic. General growth conditions necessary for the culture of bacteria can be found in texts such as BERGEY'S MANUAL OF SYSTEMATIC BACTERIOLOGY, Vol. 1, N. R. Krieg, ed., Williams and Wilkins, Baltimore/London (1984). A “host cell” can also be one in which the endogenous genes or promoters or both have been modified to produce one or more of the polypeptide components of the complex of the invention.

As used in herein “cell” is used in its usual biological sense, and does not refer to an entire multicellular organism. The cell can, for example, be in vivo, in vitro or ex vivo, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues in vitro, ex vivo, or in vivo through injection or infusion pump, with or without their incorporation in biopolymers.

In another embodiment, the invention features an enzymatic nucleic acid molecule having one or more non-nucleotide moieties, and having enzymatic activity to cleave an RNA or DNA molecule.

In a further embodiment, the described nucleic acid molecules, such as antisense or ribozymes, can be used in combination with other known treatments to treat conditions or diseases discussed above. For example, the described molecules can be used in combination with one or more known therapeutic agents.

Antisense molecules can be modified or unmodified RNA, DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). To date, the only backbone modified DNA chemistry which acts as substrates for RNase H are phosphorothioates, phosphorodithioates, and borontrifluoridates. Recently it has been reported that 2′-arabino and 2′-fluoro-arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174 which was filed on Sep. 21, 1998) all of these are incorporated by reference herein in their entirety.

Several varieties of enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83 87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakacane & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

The enzymatic nature of an enzymatic nucleic acid molecule can allow the concentration of enzymatic nucleic acid molecule necessary to affect a therapeutic treatment to be lower. This reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to greatly attenuate the catalytic activity of a enzymatic nucleic acid molecule.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieve efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Man, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

Enzymatic nucleic acid molecules of the invention that are allosterically regulated (“allozymes”) can be used to modulate PTRF or a PTRF-receptor gene expression. These allosteric enzymatic nucleic acids or allozymes (see for example George et al, U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent, which in turn modulates the activity of the enzymatic nucleic acid molecule and modulates expression of PTRF or a PTRF-receptor gene. In response to interaction with a predetermined signaling agent, the allosteric enzymatic nucleic acid molecule's activity is activated or inhibited such that the expression of a particular target is selectively down-regulated. The target can comprise PTRF or a PTRF-receptor gene.

Oligonucleotides (e.g.; antisense, GeneBlocs) are synthesized using protocols known in the art as described in Caruthers et al., 1992, Methods in Enzymology 211, 3 19, Thompson et al., International PCT Publication No. WO 99/54459, Wincott et al., 1995, Nucleic Acids Res. 23, 2677 2684, Wincott et al., 1997, Methods Mol. Bio., 74, 59, Brennan et al, 1998, Biotechnol Bioeng., 61, 33 45, and Brennan, U.S. Pat. No. 6,001,311. All of these references are incorporated herein by reference. In a non-limiting example, small scale syntheses are conducted on a 394 Applied Biosystems, Inc. synthesizer. Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-fluoro, 2′-O-methyl, 2′-H (for a review see Usman and Cedergren, 1992, TIBS 17, 34; Usman et al., 1994, Nucleic Acids Symp. Ser. 31, 163).

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211, 3-19 (incorporated by reference herein) have expanded the ability to modify nucleic acid molecules by introducing nucleotide modifications to enhance their nuclease stability as described above. The use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules and/or other chemical or biological molecules). The treatment of subjects with nucleic acid molecules can also include combinations of different types of nucleic acid molecules.

In one embodiment, nucleic acid catalysts having chemical modifications that maintain or enhance enzymatic activity are provided. Such nucleic acids are also generally more resistant to nucleases than unmodified nucleic acid.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331 417, and Mesmaeker et al., 1994, Novel Backbone Replacements for Oligonucleotides, in Carbohydrate Modifications in Antisense Research, ACS, 24 39. These references are hereby incorporated by reference herein. Various modifications to nucleic acid (e.g., antisense and ribozyme) structure can be made to enhance the utility of these molecules. For example, such modifications can enhance shelf-life, half-life in vitro, bioavailability, stability, and ease of introduction of such oligonucleotides to the target site, including e.g., enhancing penetration of cellular membranes and conferring the ability to recognize and bind to targeted cells.

Administration of Nucleic Acid Molecules. Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by a incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example, through the use of conjugates and biodegradable polymers. For a comprehensive review on drug delivery strategies including CNS delivery, see Ho et al., 1999, Cum Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400.

The negatively charged polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

Nucleic acid molecules of the invention can also be administered in the form of suppositories, e.g., for rectal administration of the drug or via a catheter directly to the bladder itself. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Alternatively, certain of the nucleic acid molecules described herein can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591 5; Kashani-Sabet et al., 1992, Antisense Res. Dev., 2, 3 15; propulic et al., 1992, J. Virol., 66, 1432 41; Weerasinghe et al., 1991, J. Virol., 65, 5531 4; Ojwang et al., 1992, Proc. Natl. Acad. Sci. USA, 89, 10802 6; Chen et al., 1992, Nucleic Acids Res., 20, 4581 9; Sarver et al., 1990 Science, 247, 1222 1225; Thompson et al, 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector.

In one aspect the description provides an expression vector comprising a nucleic acid sequence encoding at least one of the nucleic acid molecules of the instant invention. The nucleic acid sequence encoding the nucleic acid molecule of the instant invention is operably linked in a manner which allows expression of that nucleic acid molecule.

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743 7; Gao and Huang 1993, Nucleic Acids Res., 21, 2867 72; Lieber et al., 1993, Methods Enzymol., 217, 47 66; Zhou et al., 1990, Mol. Cell. Biol., 10, 4529 37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3 15; Ojwang et al., 1992, Proc. Natl. Acad. Sci. USA, 89, 10802 6; Chen et al, 1992, Nucleic Acids Res., 20, 4581 9; Yu et al., 1993, Proc. Natl. Acad. Sci. USA, 90, 6340 4; L'Huillier et al., 1992, EMBO J., 11, 4411 8; Lisziewicz et al., 1993, Proc. Natl. Acad. Sci. U.S.A, 90, 8000 4; Thompson et al., 1995, Nucleic Acids Res., 23, 2259; Sullenger & Cech, 1993, Science, 262, 1566).

In another aspect the description provides an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment, an isolated nucleic acid molecule as described herein comprises a nucleic acid molecule that is a complement of the nucleotide sequence of MG53, a MG53 binding protein, and/or a MG53 receptor. As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

Descriptions of the molecular biological techniques useful to the practice of the invention including mutagenesis, PCR, cloning, and the like include Berger and Kimmel, GUIDE TO MOLECULAR CLONING TECHNIQUES, METHODS IN ENZYMOLOGY, volume 152, Academic Press, Inc., San Diego, Calif. (Berger); Sambrook et al., MOLECULAR CLONING—A LABORATORY MANUAL (2nd Ed.), Vol. 1-3, Cold Spring Harbor Laboratory, Cold Spring Harbor, N.Y., 1989, and CURRENT PROTOCOLS IN MOLECULAR BIOLOGY, F. M. Ausubel et al., eds., Current Protocols, a joint venture between Greene Publishing Associates, Inc. and John Wiley & Sons, Inc.; Berger, Sambrook, and Ausubel, as well as Mullis et al., U.S. Pat. No. 4,683,202 (1987); PCR PROTOCOLS A GUIDE TO METHODS AND APPLICATIONS (Innis et al. eds), Academic Press, Inc., San Diego, Calif. (1990) (Innis); Arnheim & Levinson (Oct. 1, 1990) C&EN 36-47; Lueng, et al.

In yet another embodiment, a nucleic acid of the invention is expressed in mammalian cells using a mammalian expression vector. For suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

A polynucleotide can be a DNA molecule, a cDNA molecule, genomic DNA molecule, or an RNA molecule. A polynucleotide as DNA or RNA can include a sequence wherein T (thymidine) can also be U (uracil). If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are substantially complementary to each other when a sufficient number of corresponding positions in each molecule are occupied by nucleotides that can hybridize with each other in order to affect the desired process.

Transformation of a host cell with recombinant DNA may be carried out by conventional techniques as are well known to those skilled in the art. By “transformation” is meant a permanent or transient genetic change induced in a cell following incorporation of new DNA (i.e., DNA exogenous to the cell).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

In any of the embodiments, the nucleic acids encoding the PTRF or a PTRF-receptor can be present as: one or more naked DNAs; one or more nucleic acids disposed in an appropriate expression vector and maintained episomally; one or more nucleic acids incorporated into the host cell's genome; a modified version of an endogenous gene encoding the components of the complex; one or more nucleic acids in combination with one or more regulatory nucleic acid sequences; or combinations thereof. The nucleic acid may optionally comprise a linker peptide or fusion protein component, for example, His-Tag, FLAG-Tag, fluorescent protein, GST, TAT, an antibody portion, e.g., Fc, a signal peptide, and the like, at the 5′ end, the 3′ end, or at any location within the ORF.

Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂, RbCl, liposome, or liposome-protein conjugate can be used. Transformation can also be performed after forming a protoplast of the host cell or by electroporation. Thes examples are not limiting on the present invention; numerous techniques exist for transfecting host cells that are well known by those of skill in the art and which are contemplated as being within the scope of the present invention.

When the host is a eukaryote, such methods of transfection with DNA include calcium phosphate co-precipitates, conventional mechanical procedures such as microinjection, electroporation, insertion of a plasmid encased in liposomes, or virus vectors, as well as others known in the art, may be used. The eukaryotic cell may be a yeast cell (e.g., Saccharomyces cerevisiae) or may be a mammalian cell, including a human cell. For long-term, high-yield production of recombinant proteins, stable expression is preferred.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

In another aspect, the description provides a polypeptide comprising an isolated and substantially purified PTRF polypeptide (e.g., SEQ ID NOs.: 1, 2, 3, and 4). In certain embodiments, the PTRF polypeptides include an amino acid sequence that is substantially identical to the amino acid sequence of a human PTRF polypeptide (SEQ ID NO:1).

In still another aspect the invention includes a method of producing a polypeptide by culturing a cell that contains an endogenous PTRF nucleic acid disposed upstream or downstream of an exogenous promoter. In certain embodiments, the exogenous promoter is incorporated into a host cell's genome through homologous recombination, strand break or mismatch repair mechanisms.

In a further aspect, the invention provides a method for modulating the activity of a PTRF polypeptide by contacting a cell sample that includes the MG53 polypeptide with a compound that binds to the PTRF polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

In certain aspects, the description provides a therapeutic composition comprising a pharmaceutically acceptable carrier and/or excipient and an effective amount of PTRF polypeptide having at least 30%, 40%, 50%, 60%, 70%, 80%, 90% or 100% sequence identity to at least one amino acid sequence as set forth in SEQ ID NOs.: 1, 2, 3 or 4. In certain embodiments, the description provides a PTRF polypeptide analog, pseudopeptide or peptidomimetic based thereon; or a PTRF-specific antibody or anti-PTRF antigen binding fragment. As described herein, PTRF mediates the repair of damage to cellular membranes. Therefore, targeting the expression and/or activity of these nucleic acids, polypeptides, and homologs thereof will allow for a novel treatment of various acute and chronic diseases and conditions related to tissue repair. In another embodiment, the description provides a composition comprising an effective amount of a PTRF polypeptide and a pharmaceutically acceptable excipient or carrier. In an exemplary embodiment, the PTRF polypeptide is a polypeptide having at least 90% sequence identity to at least one of SEQ ID NO. 1, 2, 3, or 4, wherein the peptide is effective for repairing cell membrane damage. In a preferred embodiment, the PTRF polypeptide comprises a polypeptide as set forth in SEQ ID NO:1.

In another aspect, the description provides a PTRF polypeptide as described herein, wherein the PTRF polypeptide is joined covalently in a single, contiguous polypeptide chain with an additional polypeptide selected from the group consisting of an MG53 polypeptide, TAT polypeptide, RFP, GFP, FLAG tag, 6×His tag, maltose binding protein tag (MBP), a signal peptide, and combinations thereof.

In still another aspect, the description provides a composition for promoting the repair of a damaged cell membrane comprising a therapeutically effective amount of a polypeptide having at least 90% sequence identity to SEQ ID NO:1 and a pharmaceutically acceptable carrier or excipient, wherein the composition promotes the repair of a damaged cell membrane. In an embodiment, the polypeptide is joined covalently in a single, contiguous polypeptide chain with an additional polypeptide selected from the group consisting of an MG53 polypeptide, TAT polypeptide, RFP, GFP, FLAG tag, 6×His tag, maltose binding protein tag (MBP), a signal peptide, and combinations thereof.

In another aspect, the description provides methods for the treatment or prevention of cellular damage comprising administering a therapeutically or prophylactically effective amount of a composition comprising a PTRF polypeptide as described herein. In one embodiment, the method includes a step of identifying or diagnosing a subject as having cellular damage or a condition related to cell membrane damage or membrane repair dysfunction prior to the step of administering an effective amount of a composition as described herein. In another embodiment, the composition to be administered further comprises an MG53 polypeptide (SEQ ID NO: 9). Exemplary conditions related to cell membrane damage or membrane repair dysfunction are described herein and include, e.g., skeletal or cardiac muscle cell damage, wounds or lesions, e.g., tissue damage due to a surgical procedure. In certain embodiments, therapeutic compositions as described herein are administered locally. In other embodiments, the therapeutic composition as described herein are administered systemically.

In another aspect, the description provides a method for promoting tissue regeneration comprising administering an effective amount of a composition as described herein, in vivo or in vitro to a tissue, wherein the tissue growth and/or repair is enhanced relative to tissue growth and/or repair in the absence of the composition.

In certain other aspects, the invention includes methods for the treatment of or amelioration of tissue damage and/or disorders related to tissue damage comprising administering an effective amount of the composition of the invention to a subject in need thereof. In certain embodiments, the invention comprises methods for treating tissue damage or wounds, for example, cuts, abrasions, lesions, ulcers, burns, bed sores, gum diseases, mucositis, and the like, comprising administering an effective amount of the therapeutic composition of the invention to a subject in need thereof.

In another aspect, the invention includes a method of detecting the presence of a PTRF polypeptide in a sample. In the method, a sample is contacted with a compound that selectively binds to the polypeptide under conditions allowing for formation of a complex between the polypeptide and the compound. The complex is detected, if present, thereby identifying the PTRF polypeptide within the sample.

The invention also features antibodies and antigen-binding fragments that immunoselectively-bind to a PTRF antigen or epitope, fragments, homolog, analog, pseudopeptide, peptidomimetic or derivative thereof.

Antibodies

The term “antibody” as used herein refers to immunoglobulin molecules and immunologically active portions of immunoglobulin (Ig) molecules, i.e., molecules that contain an antigen-binding site that specifically binds (immunoreacts with) an antigen, comprising at least one, and preferably two, heavy (H) chain variable regions (abbreviated herein as VH), and at least one and preferably two light (L) chain variable regions (abbreviated herein as VL). Such antibodies include, but are not limited to, polyclonal, monoclonal, chimeric, single chain, Fab, Fab′ and F(ab′)2 fragments, and an Fab expression library. The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

Antibodies can be prepared from the intact polypeptide or fragments containing peptides of interest as the immunizing agent. A preferred antigenic polypeptide fragment is 15-100 contiguous amino acids of PTRF or a PTRF-receptor protein. In one embodiment, the peptide is located in a non-transmembrane domain of the polypeptide, e.g., in an extracellular or intracellular domain. An exemplary antibody or antibody fragment binds to an epitope that is accessible from the extracellular milieu and that alters the functionality of the protein. In certain embodiments, the present invention comprises antibodies that recognize and are specific for one or more epitopes of a PTRF or a PTRF-receptor protein, variants, portions and/or combinations thereof. In alternative embodiments antibodies of the invention may target and interfere with the PTRF/PTRF-receptor interaction to inhibit signaling.

The preparation of monoclonal antibodies is well known in the art; see for example, Harlow et al., Antibodies: A Laboratory Manual, page 726 (Cold Spring Harbor Pub. 1988). Monoclonal antibodies can be obtained by injecting mice or rabbits with a composition comprising an antigen, verifying the presence of antibody production by removing a serum sample, removing the spleen to obtain B lymphocytes, fusing the lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by techniques well known in the art.

In other embodiments, the antibody can be recombinantly produced, e.g., produced by phage display or by combinatorial methods. Phage display and combinatorial methods can be used to isolate recombinant antibodies that bind to PTRF or a PTRF-receptor proteins or fragments thereof (as described in, e.g., Ladner et al. U.S. Pat. No. 5,223,409; Fuchs et al. (1991) Bio/Technology 9:1370-1372; Hay et al. (1992) Hum Antibod Hybridomas 3:81-85; Huse et al. (1989) Science 246:1275-1281; Clackson et al. (1991) Nature 352:624-628; Gram et al. (1992) PNAS 89:3576-3580.

Human monoclonal antibodies can also be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855).

A therapeutically useful antibody to the components of the complex of the invention or the complex itself may be derived from a “humanized” monoclonal antibody. Humanized monoclonal antibodies are produced by transferring mouse complementarity determining regions from heavy and light variable chains of the mouse immunoglobulin into a human variable domain, then substituting human residues into the framework regions of the murine counterparts. The use of antibody components derived from humanized monoclonal antibodies obviates potential problems associated with immunogenicity of murine constant regions. Techniques for producing humanized monoclonal antibodies can be found in Jones et al., Nature 321: 522, 1986 and Singer et al., J. Immunol. 150: 2844, 1993. The antibodies can also be derived from human antibody fragments isolated from a combinatorial immunoglobulin library; see, for example, Barbas et al., Methods: A Companion to Methods in Enzymology 2, 119, 1991. In addition, chimeric antibodies can be obtained by splicing the genes from a mouse antibody molecule with appropriate antigen specificity together with genes from a human antibody molecule of appropriate biological specificity; see, for example, Takeda et al., Nature 314: 544-546, 1985. A chimeric antibody is one in which different portions are derived from different animal species.

Anti-idiotype technology can be used to produce monoclonal antibodies that mimic an epitope. An anti-idiotypic monoclonal antibody made to a first monoclonal antibody will have a binding domain in the hypervariable region that is the “image” of the epitope bound by the first monoclonal antibody. Alternatively, techniques used to produce single chain antibodies can be used to produce single chain antibodies. Single chain antibodies are formed by linking the heavy and light chain fragments of the Fv region via an amino acid bridge, resulting in a single chain polypeptide. Antibody fragments that recognize specific epitopes, e.g., extracellular epitopes, can be generated by techniques well known in the art. Such fragments include Fab fragments produced by proteolytic digestion, and Fab fragments generated by reducing disulfide bridges. When used for immunotherapy, the monoclonal antibodies, fragments thereof, or both may be unlabelled or labeled with a therapeutic agent. These agents can be coupled directly or indirectly to the monoclonal antibody by techniques well known in the art, and include such agents as drugs, radioisotopes, lectins and toxins.

The dosage ranges for the administration of monoclonal antibodies are large enough to produce the desired effect, and will vary with age, condition, weight, sex, age and the extent of the condition to be treated, and can readily be determined by one skilled in the art. Dosages can be about 0.1 mg/kg to about 2000 mg/kg. The monoclonal antibodies can be administered intravenously, intraperitoneally, intramuscularly, and/or subcutaneously.

In certain embodiments of the invention, at least one epitope encompassed by the antigenic peptide is a region of PTRF or a PTRF-receptor that is located on the surface of the protein, e.g., a hydrophilic region. A hydrophobicity analysis of the protein sequence will indicate which regions of a polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein. A protein of the invention, or a derivative, fragment, analog, homolog or ortholog thereof, may be utilized as an immunogen in the generation of antibodies that immunospecifically bind these protein components.

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Ban Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol. 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology, 10:779-783 (1992)); Lonberg et al. (Nature, 368:856-859 (1994)); Morrison (Nature, 368:812-13 (1994)); Fishwild et al, (Nature Biotechnology, 14:845-51 (1996)); Neuberger (Nature Biotechnology, 14:826 (1996)); and Lonberg and Huszar (Intern. Rev. Immunol., 13:65-93 (1995)).

Human antibodies may additionally be produced using transgenic nonhuman animals which are modified so as to produce fully human antibodies rather than the animal's endogenous antibodies in response to challenge by an antigen. The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096.

Fab Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., Science 246:1275-1281 (1989)) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F(ab′)2 fragment produced by pepsin digestion of an antibody molecule; (ii) an Fab fragment generated by reducing the disulfide bridges of an F(ab′)2 fragment; (iii) an Fab fragment generated by the treatment of the antibody molecule with papain and a reducing agent and (iv) Fv fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit. Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. Similar procedures are disclosed in WO 93/08829, published May 13, 1993, and Traunecker et al., EMBO J., 10:3655-3659 (1991).

Antibody variable domains with the desired binding specificities (antibody-antigen combining sites) can be fused to immunoglobulin constant domain sequences. For further details of generating bispecific antibodies see, for example, Suresh et al., Methods in Enzymology, 121:210 (1986); and Brennan et al., Science 229:81 (1985).

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The “diabody” technology described by Hollinger et al., Proc. Natl. Acad. Sci. USA 90:6444-6448 (1993) has provided an alternative mechanism for making bispecific antibody fragments. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991). Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA.

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Immunoconjugates

The invention also pertains to immunoconjugates comprising an antibody conjugated to a chemical agent, or a radioactive isotope (i.e., a radioconjugate). Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257: 286-288 (1982) via a disulfide-interchange reaction.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 500 mg/kg body weight.-Common dosing frequencies may range, for example, from twice daily to once a week.

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York. The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions. The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPO™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., Fab or F(ab)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA includes Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques intracavity, or transdermally, alone or with effector cells.

Kits

In further aspect, the description provides a kit or system utilizing any one of the methods, selection strategies, materials, or components described herein. Exemplary kits according to the present disclosure will optionally, additionally include instructions for performing methods or assays, packaging materials, one or more containers which contain an assay, a device or system components, or the like.

Pharmacological or Therapeutic Formulations and Routes of Administration

The biological molecules (e.g., nucleic acids, polypeptides, and antibodies, etc.) (also referred to herein as “active compounds”) of the invention, and derivatives, fragments, analogs and homologs thereof, can be incorporated into pharmaceutical compositions suitable for administration, and use as pharmaceutical or therapeutic agents. Pharmaceutical agents prevent, inhibit the occurrence, or treat (alleviate a symptom to some extent, preferably all of the symptoms) a disease state in a subject. The amount of active ingredient that can be combined with the carrier materials to produce a single dosage form varies depending upon the host treated and the particular mode of administration. Dosage unit forms generally contain between from about 1 mg to about 1000 mg of an active ingredient.

Such active compounds may comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

Compositions as described herein can also be administered to a subject in combination with other therapeutic compounds to increase the overall therapeutic effect, e.g., MG53 nucleic acids or polypeptides. The use of multiple compounds to treat an indication can increase the beneficial effects while reducing the presence of side effects.

The present description provides pharmaceutically acceptable formulations of the compounds described. These formulations include salts of the above compounds, e.g., acid addition salts, for example, salts of hydrochloric, hydrobromic, acetic acid, and benzene sulfonic acid. A pharmacological or therapeutic composition or formulation refers to a composition or formulation in a form suitable for administration, e.g., systemic administration, into a cell or subject, preferably a human. By “systemic administration” is meant in vivo systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

Preparations for administration of the therapeutic of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles include sodium chloride solution, Ringer's dextrose, dextrose and sodium chloride, lactated Ringer's intravenous vehicles including fluid and nutrient replenishers, electrolyte replenishers, and the like. Preservatives and other additives may be added such as, for example, antimicrobial agents, anti-oxidants, chelating agents and inert gases and the like.

A therapeutically effective dose refers to that amount of the therapeutic sufficient to result in amelioration or delay of symptoms. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the 1050 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Administration routes which lead to systemic absorption include, without limitations: intravenous, subcutaneous, intraperitoneal, inhalation, oral, intrapulmonary and intramuscular. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, intraperitoneal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

By pharmaceutically acceptable formulation is meant, a composition or formulation that allows for the effective distribution of the active compounds as described herein in the physical location most suitable for their desired activity. Non-limiting examples of agents suitable for formulation with the active compounds described herein include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of nucleic acid molecules include material described in Boado et al., 1998, J. Pharm. Sci., 87, 1308-1315; Tyler et al, 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose or pharmaceutically effective amount is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 1000 mg/kg body weight/day of active ingredients is administered dependent upon potency of the negatively charged polymer.

The formulations can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For administration by inhalation, the compounds are delivered in the form of an aerosol spray from pressured container or dispenser which contains a suitable propellant, e.g., a gas such as carbon dioxide, or a nebulizer.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™. (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., the therapeutic complex of the invention) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Additional objects and advantages of the present invention will be appreciated by one of ordinary skill in the art in light of the current description and examples of the preferred embodiments, and are expressly included within the scope of the present invention.

### EXAMPLES

MG53-Mediated Membrane Repair

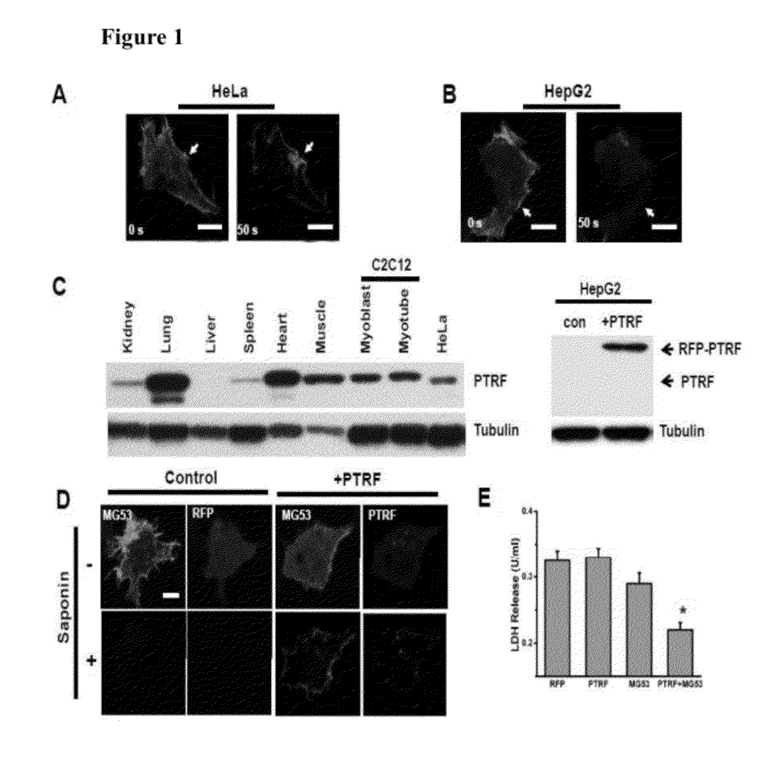

Membrane resealing is an intrinsic property of nearly all eukaryotic cells that is necessary to restore cellular integrity in response to injury and maintain homeostasis. Elevated membrane repair capacity is required in striated muscles due to the contractile nature of these cells under normal physiological conditions. While MG53 is a muscle-specific protein, it may accelerate the conserved cell membrane repair mechanisms in non-muscle cells to provide beneficial effects on the health of the targeted tissues as MG53-mediated cell membrane repair can be recapitulated in multiple non-muscle cell types, including keratinocytes, fibroblasts, and epithelial cells (Nat Cell Biol. 2009 January; 11(1):56-64). An exception was observed in HepG2 cells, a hepatocellular carcinoma cell line, which failed to show GFP-MG53 translocation to sites of membrane disruption. An example is shown in FIG. 1a, where GFP-MG53 expressed in HeLa cells displayed rapid translocation toward the mechanical injury sites for formation of a membrane repair patch. In contrast, HepG2 cells showed no accumulation of GFP-MG53 at the injury sites (FIG. 1b). One explanation for compromised GFP-MG53 nucleation at membrane injury sites is that HepG2 cells lack cellular factors essential for MG53 function.

PTRF is Required for MG53-Mediated Membrane Repair

Previous studies have shown that polymerase I and transcript release factor (PTRF) is expressed in most tissues, with the exception of liver that does not contain mRNA for PTRF (Hasegawa et al. 2000). Western blots revealed abundant expression of PTRF protein in mouse kidney, lung, heart and skeletal muscle, but not in liver tissue or HepG2 cells (FIG. 1c). To test whether PTRF contributes to MG53-mediated cell membrane repair, we transfected HepG2 cells with a red fluorescent protein labeled PTRF (RFP-PTRF) and used saponin detergent to damage the plasma membrane. As shown in FIG. 1d, GFP-MG53 expressed in HepG2 cells was diffuse and could not target to the plasma membrane upon treatment with 0.005% saponin, whereas HepG2 cells with co-expression of RFP-PTRF and GFP-MG53 displayed concentration of GFP-MG53 and RFP-PTRF at the cell membrane following treatment with saponin. This saponin-induced translocation of GFP-MG53 to the plasma membrane was similar to those observed in HEK293 cells, C2C12 myoblasts and cardiomyocytes (Cai et al. 2009a; Cao et al.; Wang et al.). Further studies tested if PTRF mediates MG53-dependent cell resealing following mechanical damage. We measured the release of an intracellular enzyme, lactate dehydrogenase (LDH), from a population of HepG2 cells damaged with glass microbeads. Expression of MG53 or PTRF alone did not improve the membrane repair capacity over the parental HepG2 cells, whereas co-expression of PTRF and MG53 led to significant reduction of LDH release from HepG2 cells (FIG. 1e), suggesting that PTRF and MG53 are both required for MG53-mediated membrane repair.

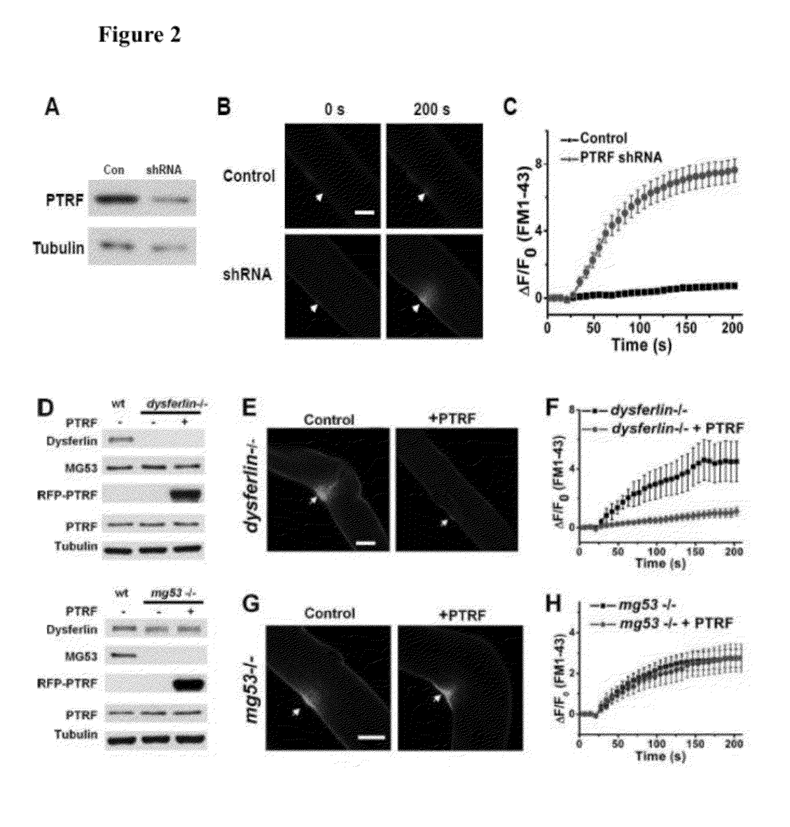

Since skeletal muscle contains abundant expression of PTRF and MG53, we used RNAi silencing to knockdown the expression of PTRF in skeletal muscle fibers to test if reduced expression of PTRF produces membrane repair defects. We generated a shRNA probe against PTRF and introduced this plasmid into the flexor digitorum brevis (FDB) muscle of viable wild type mice using electorporation (Cai et al. 2009a; Pouvreau et al. 2007). Two weeks after electroporation, western blotting was performed with transfected FDB muscles, illustrating effective knock-down of PTRF expression (FIG. 2a). For evaluation of the membrane repair capacity, individual FDB fibers were irradiated with a UV laser to cause localized damage at the sarcolemmal membrane (McNeil et al. 2003). As shown in FIG. 2b, knock down of PTRF expression led to elevated entry of FM1-43 fluorescent dye at the UV-irradiation site, indicating reduced membrane repair capacity as compared with fibers treated with a control shRNA probe. The extent of the membrane repair defects shown in FIG. 1c is similar to those observed in mg53−/−muscle (Cai et al. 2009a; Cai et al. 2009b), suggesting that reduced expression of PTRF compromises MG53 function and leads to increased susceptibility to membrane damage.

MG53 and PTRF are Both Necessary for Membrane Repair

Bansal et al showed that dysferlin contributes to membrane resealing in striated muscle cells (Bansal et al. 2003) as knockout mice for dysferlin displayed membrane repair defects in both skeletal and cardiac muscle (Bansal et al. 2003; Han et al. 2007). Our recent study showed that MG53 can interact with dysferlin to facilitate membrane repair in skeletal muscle, whereas dysferlin alone could not translocate to the acute injury site (Cai et al. 2009b). To test if increased expression of PTRF can enhance the membrane repair capacity of skeletal muscle, we overexpressed RFP-PTRF in FDB fibers from either dysferlin−/− or mg53−/−mice, since muscle fibers from both display deficient membrane repair. As shown in FIG. 2a, the expression levels of dysferlin and MG53 were comparable to wild type levels in mg53−/− and dysferlin−/−skeletal muscles, respectively. The endogenous PTRF protein levels in both dysferlin−/− and mg53−/−muscles were also comparable to the wild type control, and electroporation-induced expression of exogenous RFP-PTRF was similar in mg53−/− and dysferlin−/−muscle. While overexpression of RFP-PTRF greatly decreased the UV-irradiation induced FM1-43 dye entry in the dysferlin−/−muscle fibers (FIG. 2b, 2c), there was no significant change in mg53−/−fibers electroporated with RFP-PTRF (FIG. 2d, 2e). In both cases, these experiments used FDB fibers electroporated with RFP expression plasmid as a control. Since overexpression of PTRF can rescue the membrane repair defects in dysferlin−/−muscle but not in mg53−/−muscle, the functional role of PTRF in membrane repair likely requires the presence of MG53.

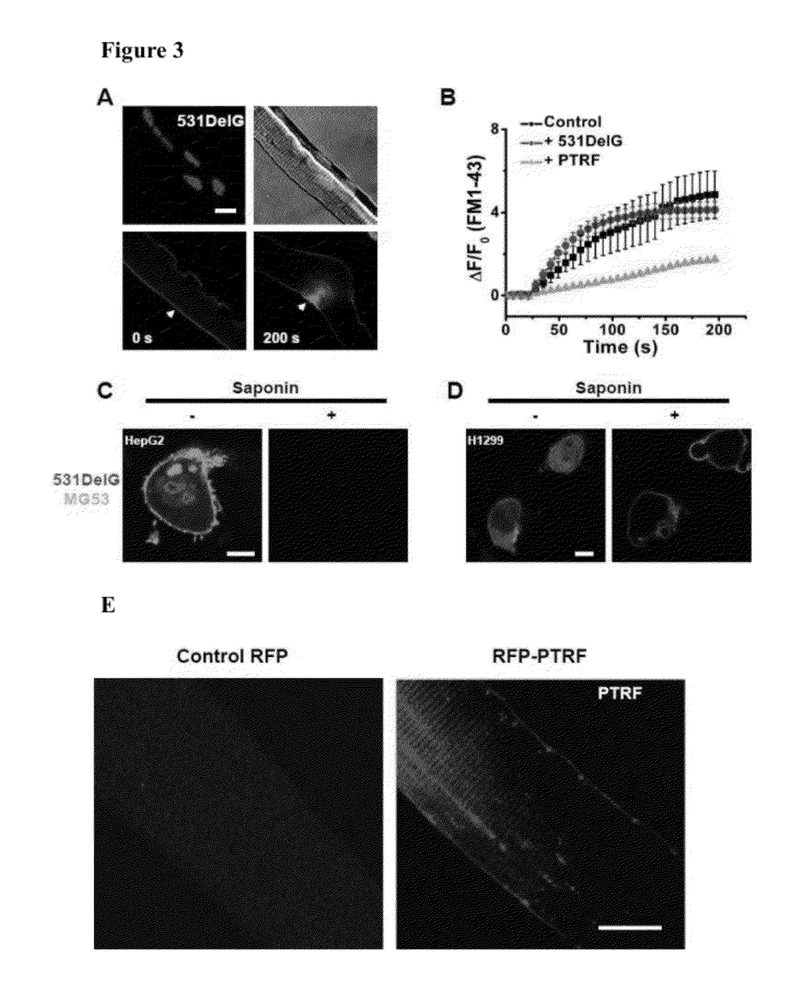

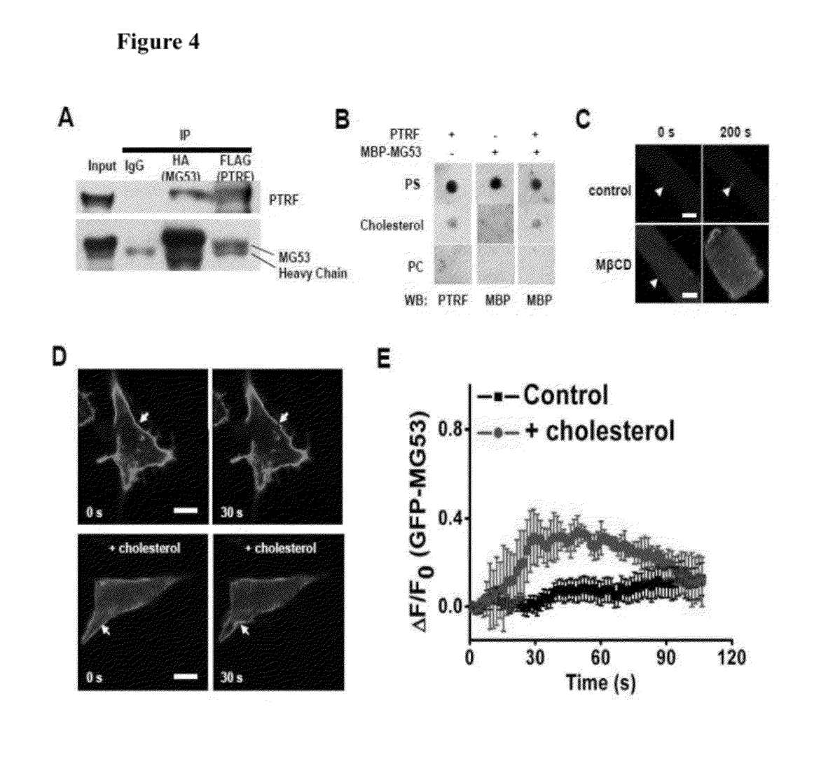

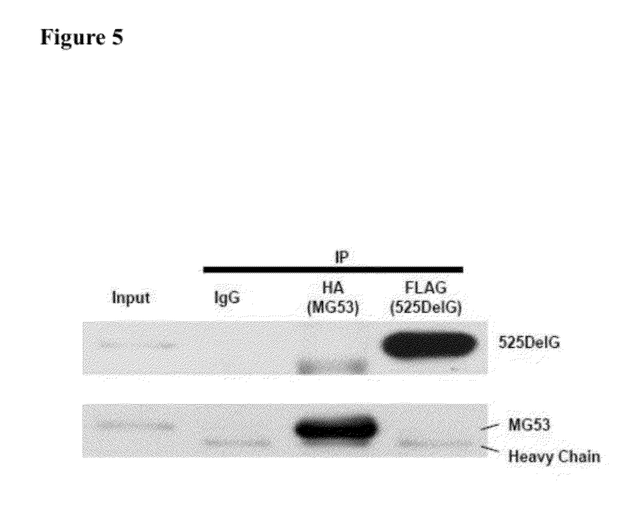

Several studies indicate mutations in PTRF lead to muscular dystrophy, cardiac disease and lipodystrophy (Dwianingsih et al.; Hayashi et al. 2009; Rajab et al.; Shastry et al.). However, no current studies address if known PTRF mutations affect membrane repair capacity. We generated the homologous mouse PTRF mutant (531DelG) for one of these know human mutations (525DelG)(Hayashi et al. 2009). The human 525DelG mutation causes a frame-shift that replaces the last 215 amino acids of PTRF with an unrelated 99 amino acid sequence, and the corresponding 531DelG mutation in the mouse gene replaces the last 215 amino acids with an unrelated 36 amino acid sequence. Similar to the human mutation (Hayashi et al. 2009), the mouse mutation also resulted in mis-location of PTRF to the nucleus (FIG. 3a, see FIG. 4 for expression of wild type PTRF in skeletal muscle), indicating that the mouse 531DelG mutation could be used as a model to study the human 525DelG function. As shown in FIGS. 3a and 3b, overexpression of the 531DelG mutant in the dysferlin−/−mice could not improve UV-irradiation induced damage to the FDB fiber, which is in sharp contrast to the significant enhancement of membrane repair function with overexpression of the wild type PTRF gene (see FIG. 2c). In vitro studies showed that 531DelG-PTRF expressed in HepG2 cells was mis-localized to the nucleus and insufficient to facilitate GFP-MG53 translocation to the plasma membrane following treatment with saponin (FIG. 3c), possibly due to altered interaction with MG53 (see FIG. 5).

We also performed similar studies with GFP-MG53 expression in H1299 cells, which lack expression of cavin-3 (McMahon et al. 2009), a different isoform of the cavin family. Normal translocation of GFP-MG53 toward the acute membrane injury site was observed in H1299 cells (FIG. 3d), indicating that PTRF (cavin-1) specifically acts with MG53 since another family member, cavin-3, is not required for MG53-mediated cell membrane repair. From this study we can speculate that one defective function with the human PTRF mutation may be linked to compromised membrane repair, thus contributing to the muscular dystrophy and cardiac complications observed in human patients.

PTRF Acts as a Membrane-Delimited Signal and Tethers MG53 to Cholesterol at the Injured Site

Our previous study showed that MG53 can detect the entry of extracellular oxidized milieu at the injury site to form a cross-linking oligomeric complex for nucleation of intracellular vesicles at the injury site (Cai et al. 2009a). In addition, we recently discovered that a cholesterol-dependent step for MG53-mediated cell membrane repair played an important role in protection of ischemia-reperfusion induced damage to cardiomyocytes (Wang et al.). Since MG53 can discriminate between intact and injured membrane, a membrane-delimited signal is likely involved in tethering of MG53 to the injured site. We have hypothesized that membrane cholesterol, which is normally embedded in the hydrophobic core of an intact membrane and is exposed during injury, could form a concentration site for recruitment of MG53-containing vesicles for membrane patch formation (Wang et al.). A challenge with this model is that MG53 itself cannot bind cholesterol (Cai et al. 2009a), thus an intermediate molecule could be involved in anchoring MG53 to the exposed cholesterol at the injury site.

One important function for PTRF is linked to caveolae biogenesis (Hill et al. 2008; Liu et al. 2008) and depletion of cholesterol from plasma membrane is known to cause disassociation of PTRF from caveolae structures (Hill et al. 2008). To test whether PTRF could anchor MG53 to cholesterol during cell membrane repair, we performed co-immunoprecipitation and found that MG53 and PTRF could physically interact with each other (FIG. 4a). The 531DelG mutation could disrupt this interaction with MG53 (FIG. 5). Using a lipid-protein overlay assay (Dowler et al. 2002), we showed that both MG53 and PTRF could interact with phosphatidylserine (PS), and neither of them could bind phosphatidylcholine (PC) (FIG. 4b), which were consistent with previous studies (Cai et al. 2009a; Hill et al. 2008). Clearly, PTRF could bind cholesterol while MG53 could not. With co-incubation of MG53 with PTRF, we could detect MG53 signal at cholesterol enriched dots, indicating that PTRF could anchor MG53 to cholesterol (FIG. 4b).

To further test the role of cholesterol in MG53-mediated cell membrane repair, we cultured C2C12 myoblasts with cholesterol present in the culture medium to enhance the cholesterol content in the plasma membrane. Membrane repair assay was performed with 10 mM DTT and 0 Ca²⁺ present in the extracellular solution, in order to test the effect of cholesterol on the Ca²⁺ and oxidation-independent component of MG53-mediated vesicle accumulation at the injury site (Cai et al. 2009a; Wang et al.). As shown in FIG. 4c, incubation of cholesterol in the culture medium led to significant enrichment of GFP-MG53 containing vesicles at the acute injury site under this experimental condition (FIG. 4d). In a separate assay, we treated wild type FDB fibers with methyl-β-cyclodextrin (MβCD) to deplete cholesterol from the sarcolemmal membrane. This MβCD treatment led to severe impact on the integrity and resealing capacity of skeletal muscle, since majority of the treated FDB fibers showed positive staining with FM1-43 dye due to reduced integrity of the sarcolemmal membrane, and none of the treated fibers could survive the damage produced by UV-irradiation (FIG. 4e).

Plasmids and Gene Transfection—Cloning of GFP-MG53, HA-MG53, and RFP-PTRF were described in detail in the supplemental material. C2C12 murine myoblast cell line, HepG2 human hepatocellular carcinoma cell line, H1299 lung cancer cell line, and HeLa human cervix carcinoma cell line were purchased from the American Type Culture Collection (ATCC) (Manassas, Va.). Cells were transfected using GeneJammer reagent per the manufacturer's directions (Stratagene).

Western Blotting and Co-immunoprecipitation—A standard protocol was used for co-immunoprecipitation of MG53 and PTRF.

In Vivo Muscle Transfection and Membrane Repair Assay—For transfection of skeletal muscle with RFP-PTRF or pU6-mRFP-shPTRF and their control vectors, 20 μg of plasmid DNA was injected into the flexor digitorum brevis (FDB) muscle following established protocols (Pouvreau, S., Royer, L., Yi, J., Brum, G., Meissner, G., Ríos, E., and Zhou, J. (2007) Proc. Natl. Acad. Sci. U.S.A. 104, 5235-5240). Experiments were performed 14 days after electroporation to allow for recovery from any damage generated during experimental manipulations (Cai, C., Masumiya, H., Weisleder, N., Matsuda, N., Nishi, M., Hwang, M., Ko, J. K., Lin, P., Thornton, A., Zhao, X., Pan, Z., Komazaki, S., Brotto, M., Takeshima, H., and Ma, J. (2009) Nat. Cell Biol. 11, 56-64). Isolated FDB fibers were irradiated with UV laser, and the entry of FM1-43 fluorescent dye (2.5 μM) into the muscle fibers was captured with a Zeiss-LSM 510 confocal microscope.

Live Cell Imaging—Confocal microscopic imaging of GFPMG53 translocation was performed following mechanical injury of the cell membrane or chemical treatment with saponin, as described previously (Wang, X., Xie, W., Zhang, Y., Lin, P., Han, L., Han, P., Wang, Y., Chen, Z., Ji, G., Zheng, M., Weisleder, N., Xiao, R. P., Takeshima, H., Ma, J., and Cheng, H. (2010) Circ. Res. 107, 76-83; Cai, C., Masumiya, H., Weisleder, N., Matsuda, N., Nishi, M., Hwang, M., Ko, J. K., Lin, P., Thornton, A., Zhao, X., Pan, Z., Komazaki, S., Brotto, M., Takeshima, H., and Ma, J. (2009) Nat. Cell Biol. 11, 56-64).

We claim:

1. A method for modulating cell membrane repair comprising administering an effective amount of a nucleic acid selected from the group consisting of:
a. an isolated and/or recombinant nucleic acid molecule having at least 90% sequence identity to at least one of SEQ ID NO. 5, 6, 7 or 8;
b. an isolated and/or recombinant nucleic acid molecule that is complementary to at least a portion of the nucleic acid of (a);
c. an isolated and/or recombinant nucleic acid molecule capable of hybridizing to at least a portion of the nucleic acid of (a);
d. an isolated and/or recombinant nucleic acid molecule capable of hybridizing to at least a portion of the nucleic acid of (b) or (c); and
e. combinations thereof.

2. The composition of claim 1, wherein the nucleic acid molecule is operably linked to a transcription regulatory nucleic acid sequence.

3. The composition of claim 2, wherein the nucleic acid molecule and transcription regulatory nucleic acid sequence are comprised within a plasmid or vector.

4. The composition of claim 3, wherein the plasmid is a bacterial plasmid.

5. The composition of claim 3, wherein the vector is a eukaryotic expression vector.

6. The composition of claim 5, wherein the vector is a viral vector.

7. The composition of claim 6, wherein the viral vector is a retroviral vector.

8. A host cell comprising the plasmid or vector of claim 3.

9. The host cell of claim 8, wherein the cell is a eukaryotic cell, and wherein the cell expresses a polypeptide encoded by the nucleic acid.

10. A method for treating a dysfunction in cell membrane repair and/or a pathological condition related to cell membrane damage in a subject comprising:
a. diagnosing or identifying a subject having a dysfunction in cell membrane repair or having a pathological condition related to cell membrane damage; and
b. administering a composition comprising a pharmaceutically acceptable carrier or excipient and an effective amount of a nucleic acid selected from the group consisting of:
i. an isolated and/or recombinant nucleic acid molecule having at least 90% sequence identity to at least one of SEQ ID NO. 5, 6, 7 or 8;
ii. an isolated and/or recombinant nucleic acid molecule that is complementary to at least a portion of the nucleic acid of (i);
iii. an isolated and/or recombinant nucleic acid molecule capable of hybridizing to at least a portion of the nucleic acid of (i);
iv. an isolated and/or recombinant nucleic acid molecule capable of hybridizing to at least a portion of the nucleic acid of (ii) or (iii); and
v. combinations thereof;
wherein the composition is effective in treating or ameliorating the dysfunction in cell membrane repair and/or the pathological condition related to cell membrane damage.

11. The method of claim 10, wherein the dysfunction in cell membrane repair and/or a pathological condition related to cell membrane damage is a member selected from the group consisting of a skin leasion, a wound, heart failure, ischemic reperfusion injury, muscular dystrophy, muscle tissue damage, diabetes, sarcopenia, an airway disorder, emphysema, acute repirature distress syndrome, age related muscle or tissue damage.

12. The method of claim 10, further including the step of co-administering at least one of a mitsugumin53 (MG53) polypeptide or a nucleic acid encoding a MG53 polypeptide or both.

13. The method of claim 10, wherein the composition is administered locally.

14. The method of claim 10, wherein the composition is administered systemically.


SEQUENCE LISTING (11 sequences)

SEQ ID NO: 1 - protein, 390 aa, Homo sapiens
  MISC_FEATURE (1)..(390): Human PTRF (accessionQ6NZI2)
MEDPTLYIVERPLPGYPDAEAPEPSSAGAQAAEEPSGAGSEELIKSDQVNGVLVLSLLDKIIGAVDQIQLTQAQLEERQAEMEGAVQSIQGELSKLGKAHATTSNTVSKLLEKVRKVSVNVKTVRGSLERQAGQIKKLEVNEAELLRRRNFKVMIYQDEVKLPAKLSISKSLKESEALPEKEGEELGEGERPEEDAAALELSSDEAVEVEEVIEESRAERIKRSGLRRVDDFKKAFSKEKMEKTKVRTRENLEKTRLKTKENLEKTRHTLEKRMNKLGTRLVPAERREKLKTSRDKLRKSFTPDHVVYARSKTAVYKVPPFTFHVKKIREGQVEVLKATEMVEVGADDDEGGAERGEAGDLRRGSSPDVHALLEITEESDAVLVDKSDSD

SEQ ID NO: 2 - protein, 392 aa, Mus musculus
  MISC_FEATURE (1)..(392): mouse PTRF (accession NP_033012)
MEDVTLHIVERPYSGFPDASSEGPEPTQGEARATEEPSGTGSDELIKSDQVNGVLVLSLLDKIIGAVDQIQLTQAQLEERQAEMEGAVQSIQGELSKLGKAHATTSNTVSKLLEKVRKVSVNVKTVRGSLERQAGQIKKLEVNEAELLRRRNFKVMIYQDEVKLPAKLSVSKSLKESEALPEKEGDELGEGERPEDDTAAIELSSDEAVEVEEVIEESRAERIKRSGLRRVDDFKKAFSKEKMEKTKVRTRENLEKTRLKTKENLEKTRHTLEKRMNKLGTRLVPVERREKLKTSRDKLRKSFTPDHVVYARSKTAVYKVPPFTFHVKKIREGEVEVLKATEMVEVGPEDDEVGAERGEATDLLRGSSPDVHTLLEITEESDAVLVDKSDSD

SEQ ID NO: 3 - protein, 392 aa, Rattus norvegicus
  MISC_FEATURE (1)..(392): rat PTRF (accessionNP_001099311)
MEDVTLHIVERPYSGYPDASSEGPEPTPGEARATEEPSGTGSDELIKSDQVNGVLVLSLLDKIIGAVDQIQLTQAQLEERQAEMEGAVQSIQGELSKLGKAHATTSNTVSKLLEKVRKVSVNVKTVRGSLERQAGQIKKLEVNEAELLRRRNFKVMIYQDEVKLPAKLSVSKSLKESEALPEKEGDELGEGERPEEDAAAIELSSDEAVEVEEVIEESRAERIKRSGLRRVDDFKKAFSKEKMEKTKVRTRENLEKTRLKTKENLEKTRHTLEKRMNKLGTRLVPVERREKLKTSRDKLRKSFTPDHVVYARSKTAVYKVPPFTFHVKKIREGEVEVLKATEMVEVGPDDDEVGAERGEATDLLRGSSPDVHTLLEITEESDAVLVDKSDSD

SEQ ID NO: 4 - protein, 390 aa, Bos taurus
  MISC_FEATURE (1)..(390): cow PTRF (accession DAA18473)
MEDTQLHIIEQPLSGYPDAGDQGSSTMGAPAAEEPSGAGSEELIKSDQVNGVLVLSLLDKIIGAVDQIQLTQAQLEERQAEMEGAVQSIQGELSKLGKAHATTSNTVSKLLEKVRKVSVNVKTVRGSLERQAGQIKKLEVNEAELLRRRNFKVMIYQDEVKLPAKVSIGKSLKESETLPEKEGDELAEGERPEEDAAALELSSDEAVEVEEVIEESRAERIKRSGLRRVDDFKKAFSKEKMEKTKVRTRENLERTRLKTKENLEKTRHTLEKRMNKLGTRLVPAERREKLKSSRDKLRKSFTPDHVVYARSKTAVYKVPPFTFHVKKIREGQVEVLKATEMVEVGAEEEEGGAERGEAADLLRGSSPDVHTLLEITEESDAVLVDKSDSD

SEQ ID NO: 5 - DNA, 1173 bp, Homo sapiens
  CDS (1)..(1173): human PTRF gene  (accession CCDS11425)
atggaggaccccacgctctatattgtcgagcggccgcttcccgggtaccccgacgccgaggccccggagccttcctccgctggggctcaggcagcggaggagccgtcgggggccggctcagaagagctgatcaagtcggaccaggtgaacggcgtgctggtgctgagcctcctggacaaaatcatcggggccgtagaccagatccagctgactcaagcacagctggaggagcggcaggcggagatggagggcgcagtgcagagcatccagggcgagctgagcaagctgggcaaggcgcacgccaccacgagcaatacggtgagcaagctgctggagaaggtgcgcaaggtcagcgtcaacgtgaagaccgtgcgcggcagcctggagcgccaggcggggcagatcaagaagctggaggtcaacgaggccgagctgctgcggcgccgcaactttaaagtcatgatctaccaggatgaagtgaagctgccggccaaactgagcatcagcaaatcgctgaaagagtcggaggcgctgccagagaaggagggcgaggagctgggcgagggcgagcggcccgaggaggacgcagcggcgctggagctttcgtcggacgaggcggtggaggttgaggaggttattgaggagtcccgcgcagagcgtatcaagcgcagcggcctgcggcgcgtggacgacttcaagaaggccttctccaaggagaagatggagaagaccaaggtgcgtacccgcgagaacctggagaagacgcgcctcaagaccaaggaaaacctggagaagacgcggcacaccctggagaagcgcatgaacaagctgggcacgcgcctggtgcccgccgagcggcgcgagaaactgaagacgtcgcgggacaagttgcgcaaatccttcacgcccgaccacgtggtgtacgcgcgctccaagaccgcggtctacaaggtgccacccttcaccttccacgtcaagaagatccgcgagggccaggtggaagtgctcaaggccaccgagatggtggaggtgggcgccgacgacgacgagggcggcgcggagcgcggggaggccggcgacctgcggcgcgggagcagccccgacgtgcacgcgctgctggagatcaccgaggagtcggacgccgtgctggtggacaagagcgacagcgactga

SEQ ID NO: 6 - protein, 390 aa, Homo sapiens; identical to SEQ ID NO: 1

SEQ ID NO: 7 - DNA, 3218 bp, Mus musculus
  misc_feature (1)..(3218): mouse PTRF gene (accession NM_008986)
acagttctggctagagtcccggtgcgcacggacgtggctcgagtttcctccgctctgggctcctgctcgctgtccagtctctcgctcctccttctctcccggtctcccgctccagttccacccggtcggccccgcacggctccgggaagccatggaggatgtcacgctccatatcgttgagcggccgtattccggatttcccgatgcttcctcagagggcccggagcccacccaaggggaggcgcgggccacggaggagccgtcggggaccggctccgacgagctgatcaagtcggaccaggtgaacggtgtgctggtgctgagccttctggataaaatcatcggcgccgttgaccagatccagctgacccaagcccagctggaggagcgacaggcggagatggagggcgctgtgcagagcatccagggagagctgagcaagctgggcaaggcgcacgccaccacgagcaacaccgtgagcaagttgctggagaaggtgcgcaaggtcagcgtcaacgtgaagaccgtgcgcggcagcctggagcgccaggccggccagataaagaaactggaggtcaacgaggcggagctgctgaggcgccgcaacttcaaagtcatgatctaccaggatgaagtcaagctgccggccaaactgagcgtcagcaagtcgctgaaagagtcggaggcactgcctgagaaggagggtgacgagctgggcgagggcgagcggcccgaggatgacaccgcggcgatcgagctgtcgtccgacgaggcggtggaggtggaggaggtgatcgaggagtcccgcgccgagcgcatcaagcgcagcggcctgcggcgcgtggacgacttcaagaaggccttctccaaggagaagatggagaagaccaaggtgcgcacgcgtgagaacctggagaagacgcgcctgaagaccaaggagaacctggagaagacacggcacacgctggagaagcgcatgaacaagctgggcacgcgcctggtgcccgtggagcgacgagagaagctgaagacatcccgggacaagctgcgcaagtccttcacgcccgaccatgtggtgtatgcgcgctccaagaccgctgtctacaaggtgccgcctttcaccttccacgtcaagaagatccgcgagggcgaggtggaggtgctgaaggccaccgagatggtggaggtgggtcccgaggacgacgaggttggcgcggagcgcggcgaggccactgacctgctgcgcgggagcagccccgacgtgcacacgctgctggagatcaccgaggagtcggacgccgtcctggtggacaagagcgacagcgactgagcaggactcgcagggctctgccctggaggccggcgcctgaccctgccgccccctgatcccccacccctgcccacaccgcctttcccttttcaaactttctcttttgcattctaagccccagtctggttgggcttttttttttttttttaaacaaaccgggggggggggaatacacccccccccccgaagagcagccctccaaagcacaggccttagaccgaggggaggtggtctctcaccagcgacagcctcaatttggacacagtctgggtaggatatgggaagaatgacccatgccctggttgtcttgatccaggatgcttttattttagctccctagttaggcccctcggcccctccccaacacagcttcatactcagctgcaccaccctacttcctgagctgtttctccttttcctcaccctgggagactccaaagcccaaatttctgcaagaggaaagagcaaatcctaaagacaacccttgtaggggcagctttctagtgccagcttgctctcccagagaggctgaggcaggagacttgtaagttcaaggcctgcctgggctgcagagtgagctcaaagccagcatgagcaatttagccagatgcagtttcaaaataagaggtggtgggggtgtatcactgatagagcccttgcttagaaggagggggtgggcaattcaaaattccacgaagataaaatgtgtgtagagagacctttgcccagaggtggcccactggaagacctgagggtctttgggggaactaaggcaatagggtgaggtaaaaggcagagtgtttggtggataaaggccccaccaaaataagcagaaggcgggtagagcctgggaaagggtggtaggtgtgtggggagaggggcttatttacaaagctacaaaacctgcacacaggagcacgtgagtattctttggaaagagagaggaagctagaagagggttgaaatggaattagaaagaaatgcccttgttaacaacacaaagaaattcaagagtcccactttggtaggatctcaagacagcagggcagcttccgggtcaggagggaagtcagttttagaaagtgtgttccaaagagacgtcagcctgattggattctctcttccagaatctaggaactgccttccgggaagccttgttttccatgcccggaaaattagggttccagcagggggcagcagcgagttgtggagaactggcagctggggaaagcaaggtgcctctggagctgagctccctagctggaagctggagcagcagggccagctggcttgcccaccctaactccctctcagctgcttctaccatggatctttctctaatgtgaaactttagagcttgccaagaccctcctgtgttccttgcctttggggccccctttacctgaaagttggggagagtggggtgctgcttcatgtccaaggcttgatttcttatagttactgaccaggttttctcctaaggacacatttgttccccctttaatggctgatcaggagcaggcaacacctcccccaattgatagctacaggctacttccttctcctcacctacatgtattccctgcttcttagaattgtagctcattgatattttggggtggggaggggatgaaaaccacaaaccttttataccatacaaagctttgctttttattttttatttttaatttttttttcttggtccccttccctcctctgaatgcttggagacggagaagctgaggagggtgggtaagtgttggatgaggtcctctgtgctgatgggctaatgttgtgggcagatgcagtttcctgtgggctctaggggagtcccttgagttgctgtgttctggtgagcagccggaccaataaacctgcttttctaaaa

SEQ ID NO: 8 - DNA, 1393 bp, Rattus norvegicus
  misc_feature (1)..(1393): rat PTRF gene (accession NM_001105841)
tcctccgctctaggctcctgctcgctttcccctctctcgctcctccttctctccgggtctcccgctccagttccaccgagtcggccccgcacggctccgggaagccatggaggatgtcacgctccatatcgtcgagcggccgtattccggatatcccgatgcttcctccgagggcccggagcccaccccaggggaggcacgggccaccgaggagccgtcggggaccggttccgacgagctgatcaagtcggaccaggtaaacggtgtactggtgctgagccttctggataaaatcatcggcgccgttgaccagatccagctgacccaagcccagctggaagagcgacaggcagagatggagggcgctgtgcagagcatccagggagagctgagcaagctgggcaaggcgcacgccaccaccagcaacaccgtgagcaagttgctggagaaggtgcgcaaggtcagcgtcaacgtgaagacggtgcgaggcagcctggagcgccaggctggccagataaagaaactggaggtcaacgaggcggagctgctgcggcgccgcaacttcaaagtcatgatctaccaggatgaagtcaagctgccggccaaactgagcgtcagcaagtctctgaaagagtcggaggcgctgccagagaaggagggcgatgagctgggcgagggcgagcggccggaggaggacgctgcggcgatcgagctgtcgtctgacgaggcggtggaggtggaggaggtgatcgaggagtcccgcgccgagcgcatcaagcgcagcggcctgcggcgcgtggacgacttcaagaaggccttctccaaggagaagatggagaagaccaaggtgcgcacgcgcgagaacctggagaagacgcgcctgaagactaaggagaacctggagaagacgcggcacacgctggagaagcgcatgaacaagctgggcacgcgcctggtgcccgtggagcgacgcgagaagctgaagacgtcgcgcgacaagctgcgcaagtccttcacgcccgaccacgtggtgtacgcgcgctccaagaccgcggtctacaaggtgccgccctttaccttccatgtcaagaagatccgcgagggcgaggtggaggtgctgaaggccaccgagatggtggaggtgggtcctgatgacgacgaggttggcgcggagcgcggcgaggccactgacctgctgcgcgggagcagccccgacgtgcacacgctgctggagatcaccgaggaatcggacgccgtcctggtggacaagagcgacagcgactgagcaggactcacggggctctgccctggaggccggtgcctgatcctgtcctccctgacccccactcctgcccaccgcctttctcatttcaaactttctcttttgcattct

SEQ ID NO: 9 - DNA, 2254 bp, Bos taurus
  misc_feature (1)..(2254): cow PTRFgene (accession BT029865)
tcttctccgggtctcccgttcccgcttcagctccatccggccggccccgcacggctccgggcagccatggaggacacccagctccacatcatcgagcagccgctttccgggtaccccgacgccggggaccaggggtcctccaccatgggggctccggcggccgaggagccgtcgggggccggttctgaggagctgatcaaatcagaccaggtgaacggcgtgctggtactgagccttctggacaaaatcatcggcgccgtcgaccagatccaactgacccaagctcagctggaggagcggcaggcggagatggagggcgccgtgcagagcatccaaggcgagctgagcaagctgggcaaggcgcacgccaccacgagcaacaccgtgagcaagttgctggagaaggtgcgcaaggtcagcgtcaacgtgaagaccgtgcgcggcagcctggagcgccaggccggccagatcaagaagctggaggtcaacgaggccgagctgctgaggcgccgcaactttaaagtcatgatctaccagcccaagaagaagctgaatgcaccctcagaccgggccacacccaagatcgtgccttttgatcttctgcctgagactgagccttttgacatgaagcctgagacagtgcctgacctcccaccctttgaccagacgcctaggaccctgccctattacccagagcccactgggcctctgcccacccctgaggagccgacagccggtgggggtgcttagggtctcctggctcacgtggaggggccgtgggcgtggcatgtcatgaatccagtccagctcactgcttagcagcggccagtctggaatggccagtctcttctaagccactgcctcttgtgagtgggtgacgggtactgtgcaaacactcgcattatggaatgttcaggcaatctggtgggcggtcatgggaaagggcgctcctctacctcttggctgccatcttaatagtagatgctccagacaccatacctgagttgtacccactgctgcctgcatgagattgcctgcaacttccatgcttctggttttttcttatttaattggaagacaattactttacaatattgtgatgttttgccatacctcagcacgaattggccataggtatacatgtgtcccctccatcctgaaccctgcccaggcttctgattgaccttccccccacttccacctcctcctcagggacaggccagtttggctcgagggccatttttacagaggaccctcatgaggcccaggccaggggtctgttctgtgccctttccatgcccctccctgcagctggggaaaacttttacacttattttttccaggttttttttttttttttttccagaaaatgttttgcaaagagatcccttatgtttgccagatcctgcctttggcagggcacccaaaagctgccaactcagagaggccaggagggggtacacttttgctggatctggcctacctatggcacagtgtttctccatttttttttttttcacatgatcatttttctaaggagtccttttagacatttttcttcctaattgtcctcttccccatgaaattttaacactatatgtatcctatatatacctggactcccccaacaccccacccaagaaccaatttccacccactttggggcaacatcctcccacatgctagagtgtcatgctagagcagaaaaaaaggtcccttctgccttcagtgtgtctcacccccttgtcctgaccacctaaagtagaggtcccgagtttttaagacttccctatctctcacctctcccctagtctgtaccctcctctgacacacagatcccaggtctccgaatgacctctccagtatcttctctggtcattccaggcagtggttgggaacagaactggctagctctaagtcaagaaagaccctacccagagccatatcatacgtgtcccttctccctgacaaatgtgaagtgcctggccaaggaccaggcacgaagtaggtgctcgagtaaatcacataaaaatcttcctaaggcagaggagcttaagtttatatagtgcggttggggagaggagaggaaggggctggaagctgtcttatggctcctcttaaattcatcccagactggtgctttcctgggttagggagctgtgtgtgcttggtggggggtcggggcggtggggagacaatggaagctgtgcatgcttttgttcagacctggtcattaaagctaactctggaaacc

SEQ ID NO: 10 - protein, 477 aa, Homo sapiens
  MISC_FEATURE (1)..(477): human MG53
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDCEAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 11 - DNA, 1434 bp, Homo sapiens
  misc_feature (1)..(1434): human MG53 gene
atgtcggctgcgcccggcctcctgcaccaggagctgtcctgcccgctgtgcctgcagctgttcgacgcgcccgtgacagccgagtgcggccacagtttctgccgcgcctgcctaggccgcgtggccggggagccggcggcggatggcaccgttctctgcccctgctgccaggcccccacgcggccgcaggcactcagcaccaacctgcagctggcgcgcctggtggaggggctggcccaggtgccgcagggccactgcgaggagcacctggacccgctgagcatctactgcgagcaggaccgcgcgctggtgtgcggagtgtgcgcctcactcggctcgcaccgcggtcatcgcctcctgcctgccgccgaggcccacgcacgcctcaagacacagctgccacagcagaaactgcagctgcaggaggcatgcatgcgtaaggagaagagtgtggctgtgctggagcatcagctggtggaggtggaggagacagtgcgtcagttccggggggccgtgggggagcagctgggcaagatgcgggtgttcctggctgcactggagggctccttggactgcgaggcagagcgtgtacggggtgaggcaggggtcgccttgcgccgggagctggggagcctgaactcttacctggagcagctgcggcagatggagaaggtcctggaggaggtggcggacaagccgcagactgagttcctcatgaaatactgcctggtgaccagcaggctgcagaagatcctggcagagtctcccccacccgcccgtctggacatccagctgccaattatctcagatgacttcaaattccaggtgtggaggaagatgttccgggctctgatgccagcgctggaggagctgacctttgacccgagctctgcgcacccgagcctggtggtgtcttcctctggccgccgcgtggagtgctcggagcagaaggcgccgccggccggggaggacccgcgccagttcgacaaggcggtggcggtggtggcgcaccagcagctctccgagggcgagcactactgggaggtggatgttggcgacaagccgcgctgggcgctgggcgtgatcgcggccgaggccccccgccgcgggcgcctgcacgcggtgccctcgcagggcctgtggctgctggggctgcgcgagggcaagatcctggaggcacacgtggaggccaaggagccgcgcgctctgcgcagccccgagaggcggcccacgcgcattggcctttacctgagcttcggcgacggcgtcctctccttctacgatgccagcgacgccgacgcgctcgtgccgctttttgccttccacgagcgcctgcccaggcccgtgtaccccttcttcgacgtgtgctggcacgacaagggcaagaatgcccagccgctgctgctcgtgggtcccgaaggcgccgaggcctga